(19) United States
(12) Patent Application Publication
(10) Pub. No.: US 2025/0003001 A1
(43) Pub. Date: Jan. 2, 2025

(54) COMPOSITIONS AND METHODS FOR IDENTIFYING TRANSPLANT REJECTION OR THE RISK THEREOF

(71) Applicant: INOVA HEALTH CARE SERVICES, Falls Church, VA (US)

(72) Inventor: Palak SHAH, McLean, VA (US)

(73) Assignee: INOVA HEALTH CARE SERVICES, Falls Church, VA (US)

(21) Appl. No.: 18/694,767

(22) PCT Filed: Sep. 22, 2022
(86) PCT No.: PCT/US2022/044426

§ 371 Date: Mar. 22, 2024

Related U.S. Application Data
(60) Provisional application No. 63/283,053, filed on Nov. 24, 2021.

(51) Int. Cl. C12Q 1/6883

(52) U.S. Cl. CPC C12Q 1/6883; C12Q 2600/158; C12Q 2600/178

(57) ABSTRACT

Provided herein are microfluidic arrays and miR panels useful in the identification of human subjects experiencing or at risk of experiencing an acute heart allograft rejection. Further provided are methods of identifying human subjects experiencing or at risk of experiencing an acute heart allograft rejection, including Acute Cellular Rejection (ACR) or antibody-mediated rejection (AMR). Further provided are system, apparatus, device, method and/or computer program product aspects, and/or combinations and sub-combinations thereof, for detecting an allograft rejection.

### GOVERNMENT SUPPORT

This invention was made with government support under 1K23HL143179 awarded by the National Institutes of Health (NIH). The government has certain rights in the invention.

### FIELD OF THE DISCLOSURE

Aspects of the present disclosure relate to devices and methods of using the same for identifying a subject experiencing or at risk of experiencing acute heart allograft rejection following heart transplantation. Some aspects of the present disclosure are directed to methods of identifying biomarkers that correlate with transplant rejection or the risk thereof.

### BACKGROUND

Heart transplantation remains the definitive therapy for patients with advanced heart failure (HF) and medically refractory symptoms. The median survival after heart transplant is 12.5 years, with only a modest improvement in survival over the past two decades.

Both acute and chronic, allograft rejection is a leading cause of morbidity after heart transplantation and leads to graft dysfunction and death. Rejection has an incidence of 10-20% in the first year after transplant but is often initially asymptomatic, requiring routine surveillance with endomyocardial biopsies (EMB). The standard of care at many centers to screen for allograft rejection remains the EMB, and in the first-year post-transplant, the average heart transplant recipient is subject to ˜10-17 biopsies. Moreover, there are ˜4,000 heart transplants in the US per year (46,000 worldwide). This number increases drastically when other organ transplants are included.

However, only 5% of EMB show evidence of allograft rejection. Additionally, there is ˜30% variability between pathologists in grading rejection within histologic sections derived from the EMB. Moreover, distinguishing the two major subtypes of rejection (acute cellular rejection (ACR) and antibody-mediated rejection (AMR)) can be challenging in certain cases, and accurate diagnosis of AMR is critical as it has distinct management implications, a higher recurrence rate, and poorer long-term prognosis when compared to ACR.

Moreover, biopsy results may have a turn-around time of 48-72 hours. This leads to delays in starting treatment, and the patient may start a non-specific therapy while awaiting biopsy results.

Other conventional methods to detect ACR or AMR are also flawed. For example, current biomarkers include gene expression profiling (GEP), soluble protein biomarkers, donor-derived cell-free DNA (dd-cfDNA), and T-cell immune function assays. Commercially available gene-expression profiling (GEP) involves the measurement of 11 mRNA transcripts implicated in immune system function. However, patients managed with GEP still require surveillance endomyocardial biopsies. Moreover, wide-scale implementation and reliance on GEP testing has been limited by its poor positive predictive value (PPV) value (˜10%) and inability to detect AMR. More recently, through sequencing of a panel of single-nucleotide polymorphisms (SNPs) in circulating, cell-free DNA, SNP mismatches in donor and recipient DNA can be used to quantify the donor-derived portion of cell-free DNA (dd-cfDNA). However, a critical limitation is that dd-cfDNA in its current application cannot accurately discriminate between ACR and AMR, and an EMB is still required.

### SUMMARY OF THE DISCLOSURE

Some aspects of the present disclosure are directed to a microfluidic array comprising one or more RNA-hybridization probes, wherein at least one of the RNA-hybridization probes hybridizes to an miR selected from the group consisting of miR-223-3p, miR-361-3p, miR-3615, miR-24-3p, miR-182-5p, miR-374a-5p, miR-23a-3p, miR-30e-5p, miR-582-3p, miR-130b-3p, miR-326 92, miR-1299, miR-23a-3p, miR-145-5p, miR-1249-3p, miR-27a-3p, miR-215-5p, miR-145-3p, miR-10b-5p, miR-582-3p, let-7b-3p, miR-142-3p, miR-450b-5p, miR-140-5p, miR-374a-5p, miR-17-5p, miR-143-3p, miR-130b-3p, miR-1-3p, miR-542-3p, miR-484, miR-345-5p, miR-125a-5p, miR-338-5p, miR-769-5p, miR-193a-5p, miR-454-3p, miR-223-5p, and let-7d-3p.

Some aspects of the present disclosure are directed to a microfluidic array comprising one or more RNA-hybridization probes, wherein at least one of the RNA-hybridization probes hybridizes to an miR selected from the group consisting of miR-30e-5p, let-7g-5p, miR-223-3p, miR-3615, miR-374a-5p, miR-182-5p, miR-345-5p, miR-3⁶¹-3p, miR-130b-3p, miR-1299, miR-376c-3p, miR-326, and any combination thereof.

In some aspects, the microfluidic array comprises an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, an RNA-hybridization probe that hybridizes to miR-326.

Some aspects of the present disclosure are directed to a microfluidic array comprising one or more RNA-hybridization probes, wherein at least one of the RNA-hybridization probes hybridizes to an miR selected from the group consisting of miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, miR-589-5p, and any combination thereof.

In some aspects, the microfluidic array comprises an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to miR-589-5p.

Some aspects of the present disclosure are directed to a fluidic chip comprising one or more RNA-hybridization probes, wherein at least one of the RNA-hybridization probes hybridizes to an miR selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326, and any combination thereof.

In some aspects, the fluidic chip comprises an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, an RNA-hybridization probe that hybridizes to miR-326.

Some aspects of the present disclosure are directed to a fluidic chip comprising one or more RNA-hybridization probes, wherein at least one of the RNA-hybridization probes hybridizes to an miR selected from the group consisting of miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-3³¹-5p, miR-140-5p, miR-223-5p, miR-582-3p, miR-122-3p, miR-589-5p, and any combination thereof.

In some aspects, the fluidic chip comprises an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, an RNA-hybridization probe that hybridizes to and miR-589-5p.

In some aspects, the fluidic chip is a microfluidic chip.

Some aspects of the present disclosure are directed to a panel of RNA-hybridization probes that hybridize to one or more miR selected from the group consisting of miR-223-3p, miR-361-3p, miR-3615, miR-24-3p, miR-182-5p, miR-374a-5p, miR-23a-3p, miR-30e-5p, miR-582-3p, miR-130b-3p, miR-326 92, miR-1299, miR-23a-3p, miR-145-5p, miR-1249-3p, miR-27a-3p, miR-215-5p, miR-145-3p, miR-10b-5p, miR-582-3p, let-7b-3p, miR-142-3p, miR-450b-5p, miR-140-5p, miR-374a-5p, miR-17-5p, miR-143-3p, miR-130b-3p, miR-1-3p, miR-542-3p, miR-484, miR-345-5p, miR-125a-5p, miR-338-5p, miR-769-5p, miR-193a-5p, miR-454-3p, miR-223-5p, let-7d-3p, and any combination thereof for use in identifying a human subject afflicted with or at risk of developing an acute heart allograft rejection following a heart transplant.

In some aspects, the acute heart allograft rejection comprises ACR, AMR, or a combination thereof.

Some aspects of the present disclosure are directed to a panel of RNA-hybridization probes that hybridize one or more miR selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326, and any combination thereof for use in identifying a human subject afflicted with or at risk of developing ACR following a heart transplant.

In some aspects, the panel of RNA-hybridization probes comprises at least two, at least three, at least four, at least five, at least six, at least seven, at least eight, at least nine, at least ten, or at least eleven RNA-hybridization probes selected from the group consisting of an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, an RNA-hybridization probe that hybridizes to miR-326, and any combination thereof.

In some aspects, the panel of RNA-hybridization probes comprises an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326.

In some aspects, the panel of RNA-hybridization probes consists of an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326.

Some aspects of the present disclosure are directed to a panel of RNA-hybridization probes that hybridize one or more miR selected from the group consisting of miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, miR-589-5p, and any combination thereof for use in identifying a human subject afflicted with or at risk of developing AMR following a heart transplant.

In some aspects, the panel of RNA-hybridization probes comprises at least two, at least three, at least four, at least five, at least six, at least seven, at least eight, at least nine, at least ten, or at least eleven mRNAs selected from the group consisting of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, an RNA-hybridization probe that hybridizes to and miR-589-5p, and any combination thereof.

In some aspects, the panel of RNA-hybridization probes comprises an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, an RNA-hybridization probe that hybridizes to and miR-589-5p.

In some aspects, the panel of RNA-hybridization probes consists of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, an RNA-hybridization probe that hybridizes to and miR-589-5p.

Some aspects of the present disclosure are directed to a method of identifying a human subject experiencing or at risk of experiencing an acute heart allograft rejection comprising an acute cellular rejection (ACR) following a heart transplant comprising: obtaining a biological sample from the human subject, wherein the biological sample comprises RNA, contacting the biological sample comprising the RNA with a microfluidic array disclosed herein, a chip disclosed herein, or a panel disclosed herein; and determining an ACR signature score according to the following formula: ACR signature score=251.89−(a)*ln [miR-30e-5p]−(b)*ln [let-7g-5p]−(c)*ln [miR-223-3p]+(d)*ln [miR-3615]+(e)*ln [miR-374a-5p]+(f)*ln [miR-182-5p]−(g)*ln [miR-345-5p]+(h)*ln [miR-361-3p]−(i)*ln [miR-130b-3p]−(j)*ln [miR-1299]−(k)*ln [miR-376c-3p]−(l)*ln [miR-326] wherein: (a)=any number from 23 and 33; (b)=any number from 0.14 and 0.24; (c)=any number from 2.5 and 7.5; (d)=any number from 2 and 6; (e)=any number from 4 and 9; (f)=any number from 2 and 6; (g)=any number from 0.5 and 5; (h)=any number from 22 and 32; (i)=any number from 1 and 5; (j)=any number from 3 and 10; (k)=any number from 4 and 11; and (l)=any number from 6 and 14; wherein “[X]” refers to the amount of “X” in the biological sample and “Ln” represents the natural log; wherein the human subject is identified as experiencing or at risk of experiencing an acute heart allograft rejection comprising an ACR if the ACR signature score is equal to or higher than about 65.

Some aspects of the present disclosure are directed to a method of identifying a human subject experiencing or at risk of experiencing an acute heart allograft rejection comprising an acute cellular rejection (ACR) following a heart transplant comprising: obtaining a biological sample from the human subject, wherein the biological sample comprises RNA; measuring the level of a panel of miRs in the biological sample, wherein the panel of miRs comprises miR-30e-5p, let-7g-5p, miR-223-3p, miR-3615, miR-374a-5p, miR-182-5p, miR-345-5p, miR-361-3p, miR-130b-3p, miR-1299, miR-376c-3p, and miR-326; and determining an ACR signature score according to the following formula: ACR signature score=251.89−(a)*ln [miR-30e-5p]−(b)*ln [let-7g-5p]−(c)*ln [miR-223-3p]+(d)*ln [miR-3615]+(e)*ln [miR-374a-5p]+(f)*ln [miR-182-5p]−(g)*ln [miR-345-5p]+(h)*ln [miR-361-3p]−(i)*ln [miR-130b-3p]−(j)*ln [miR-1299]−(k)*ln [miR-376c-3p]−(l)*ln [miR-326] wherein: (a)=any number from 23 and 33; (b)=any number from 0.14 and 0.24; (c)=any number from 2.5 and 7.5; (d)=any number from 2 and 6; (e)=any number from 4 and 9; (f)=any number from 2 and 6; (g)=any number from 0.5 and 5; (h)=any number from 22 and 32; (i)=any number from 1 and 5; (j)=any number from 3 and 10; (k)=any number from 4 and 11; and (l)=any number from 6 and 14; wherein “[X]” refers to the amount of “X” in the biological sample, and “Ln” represents the natural log; wherein the human subject is identified as experiencing or at risk of experiencing an acute heart allograft rejection comprising an ACR if the ACR signature score is equal to or higher than about 65.

Some aspects of the present disclosure are directed to a method of diagnosing an acute heart allograft rejection comprising an ACR after a heart transplant in a human subject, comprising obtaining a biological sample from the human subject, wherein the biological sample comprises RNA; measuring the level of a panel of miRs in the biological sample, wherein the panel of miRs comprises miR-30e-5p, let-7g-5p, miR-223-3p, miR-3615, miR-374a-5p, miR-182-5p, miR-345-5p, miR-361-3p, miR-130b-3p, miR-1299, miR-376c-3p, and miR-326; and determining an ACR signature score according to the following formula: ACR signature score=251.89−(a)*ln [miR-30e-5p]−(b)*ln [let-7g-5p]−(c)*ln [miR-223-3p]+(d)*ln [miR-3615]+(e)*ln [miR-374a-5p]+(f)*ln [miR-182-5p]−(g)*ln [miR-345-5p]+(h)*ln [miR-361-3p]−(i)*ln [miR-130b-3p]−(j)*ln [miR-1299]−(k)*ln [miR-376c-3p]−(l)*ln [miR-326] wherein: (a)=any number from 23 and 33; (b)=any number from 0.14 and 0.24; (c)=any number from 2.5 and 7.5; (d)=any number from 2 and 6; (e)=any number from 4 and 9; (f)=any number from 2 and 6; (g)=any number from 0.5 and 5; (h)=any number from 22 and 32; (i)=any number from 1 and 5; (j)=any number from 3 and 10; (k)=any number from 4 and 11; and (l)=any number from 6 and 14; wherein “[X]” refers to the amount of “X” in the biological sample, and “Ln” represents the natural log; wherein the human subject is identified as experiencing or at risk of experiencing an acute heart allograft rejection comprising an ACR if the ACR signature score is equal to or higher than about 65.

In some aspects, the method further comprises isolating RNA from the biological sample. In some aspects, the RNA comprises one or more miRs.

Some aspects of the present disclosure are directed to a method of identifying a human subject experiencing or at risk of experiencing an acute heart allograft rejection comprising an ACR following a heart transplant comprising measuring the level of a panel of miRs in a biological sample obtained from the human subject, wherein the panel of miRs comprises miR-30e-5p, let-7g-5p, miR-223-3p, miR-3615, miR-374a-5p, miR-182-5p, miR-345-5p, miR-361-3p, miR-130b-3p, miR-1299, miR-376c-3p, and miR-326; and determining an ACR signature score according to the following formula: ACR signature score=251.89−(a)*ln [miR-30e-5p]−(b)*ln [let-7g-5p]−(c)*ln [miR-223-3p]+(d)*ln [miR-3615]+(e)*ln [miR-374a-5p]+(f)*ln [miR-182-5p]−(g)*ln [miR-345-5p]+(h)*ln [miR-361-3p]−(i)*ln [miR-130b-3p]−(i)*ln [miR-1299]−(k)*ln [miR-376c-3p]−(l)*ln [miR-326] wherein: (a)=any number from 23 and 33; (b)=any number from 0.14 and 0.24; (c)=any number from 2.5 and 7.5; (d)=any number from 2 and 6; (e)=any number from 4 and 9; (f)=any number from 2 and 6; (g)=any number from 0.5 and 5; (h)=any number from 22 and 32; (i)=any number from 1 and 5; (j)=any number from 3 and 10; (k)=any number from 4 and 11; and (l)=any number from 6 and 14; and wherein “[X]” refers to the amount of “X” in the biological sample, and “Ln” represents the natural log; wherein the human subject is identified as experiencing or at risk of experiencing an acute heart allograft rejection comprising an ACR if the ACR signature score is equal to or higher than about 65.

Some aspects of the present disclosure are directed to a method of diagnosing an acute heart allograft rejection comprising an ACR after a heart transplant in a human subject, comprising measuring the level of a panel of miRs in a biological sample obtained from the human subject, wherein the panel of miRs comprises miR-30e-5p, let-7g-5p, miR-223-3p, miR-3615, miR-374a-5p, miR-182-5p, miR-345-5p, miR-361-3p, miR-130b-3p, miR-1299, miR-376c-3p, and miR-326; and determining an ACR signature score according to the following formula: ACR signature score=251.89−(a)*ln [miR-30e-5p]−(b)*ln [let-7g-5p]−(c)*ln [miR-223-3p]+(d)*ln [miR-3615]+(e)*ln [miR-374a-5p]+(f)*ln [miR-182-5p]−(g)*ln [miR-345-5p]+(h)*ln [miR-361-3p]−(i)*ln [miR-130b-3p]−(j)*ln [miR-1299]−(k)*ln [miR-376c-3p]−(l)*ln [miR-326] wherein: (a)=any number from 23 and 33; (b)=any number from 0.14 and 0.24; (c)=any number from 2.5 and 7.5; (d)=any number from 2 and 6; (e)=any number from 4 and 9; (f)=any number from 2 and 6; (g)=any number from 0.5 and 5; (h)=any number from 22 and 32; (i)=any number from 1 and 5; (j)=any number from 3 and 10; (k)=any number from 4 and 11; and (l)=any number from 6 and 14; and wherein “[X]” refers to the amount of “X” in the biological sample, and “Ln” represents the natural log; wherein acute heart allograft rejection comprising an ACR is diagnosed if the ACR signature score is higher than about 65.

Some aspects of the present disclosure are directed to a method of treating an acute heart allograft rejection comprising an ACR in a human subject in need thereof, comprising measuring the level of a panel of miRs in a biological sample obtained from the human subject, wherein the panel of miRs comprises miR-30e-5p, let-7g-5p, miR-223-3p, miR-3615, miR-374a-5p, miR-182-5p, miR-345-5p, miR-361-3p, miR-130b-3p, miR-1299, miR-376c-3p, and miR-326; determining an ACR signature score according to the following formula: ACR signature score=251.89−(a)*ln [miR-30e-5p]−(b)*ln [let-7g-5p]−(c)*ln [miR-223-3p]+(d)*ln [miR-3615]+(e)*ln [miR-374a-5p]+(f)*ln [miR-182-5p]−(g)*ln [miR-345-5p]+(h)*ln [miR-361-3p]−(i)*ln [miR-130b-3p]−(j)*ln [miR-1299]−(k)*ln [miR-376c-3p]−(l)*ln [miR-326] wherein: (a)=any number from 23 and 33; (b)=any number from 0.14 and 0.24; (c)=any number from 2.5 and 7.5; (d)=any number from 2 and 6; (e)=any number from 4 and 9; (f)=any number from 2 and 6; (g)=any number from 0.5 and 5; (h)=any number from 22 and 32; (i)=any number from 1 and 5; (j)=any number from 3 and 10; (k)=any number from 4 and 11; and (l)=any number from 6 and 14; and administering an immunosuppressive therapy to the human subject identified as having an ACR signature score is equal to or higher than about 65; wherein “[X]” refers to the amount of “X” in the biological sample, and “Ln” represents the natural log.

In some aspects, (a)=any number from 26 to 31; (b)=any number from 0.16 to 0.21; (c)=any number from 4 to 6; (d)=any number from 3 to 5; (e)=any number from 6 to 7.5; (f)=any number from 3 to 5; (g)=any number from 1 to 3; (h)=any number from 25 to 28; (i)=any number from 2 to 4; (j)=any number from 5 to 7; (k)=any number from 7 to 9; and (l)=any number from 10 to 12.

In some aspects, (a)=about 28.90; (b)=about 0.19; (c)=about 5.46; (d)=about 4.77; (e)=about 6.41; (f)=about 4.41; (g)=about 2.20; (h)=about 27.69; (i)=about 3.05; (j)=about 6.17; (k)=about 7.71; and (l)=about 10.63.

In some aspects, the miR panel further comprises one or more additional miR.

In some aspects, the method further comprises administering to the human subject an immunosuppressive therapy. In some aspects, the immunosuppressive therapy comprises administering a therapy selected from the group consisting of a corticosteroid, an anti-thymocyte globulin, tacrolimus, cyclosporine, sirolimus, everolimus, myocophenolate mofeitil, azathioprine, tocilizumab, belatacept, and any combination thereof.

Some aspects of the present disclosure are directed to a method of identifying a human subject experiencing or at risk of experiencing an acute heart allograft rejection comprising an antibody-mediated rejection (AMR) following a heart transplant comprising: obtaining a biological sample from the human subject, wherein the biological sample comprises RNA; contacting the biological sample comprising the RNA with a microfluidic array disclosed herein, a chip disclosed herein, or a panel disclosed herein; and determining an AMR signature score according to the following formula: AMR signature score=222.41−(a)*ln [miR-23a-3p]−(b)*ln [miR-484]−(c)*ln [miR-340-5p]+(d)*ln [miR-193a-5p]−(e)*ln [miR-215-5p]+(f)*ln [miR-142-3p]−(g)*ln [miR-374a-5p]+(h)*ln [miR-1307]+(i)*ln [miR-185-3p]+(j)*ln [miR-4433b-3p]+(k)*ln [miR-130b-3p]+(1)*ln [miR-331-5p]+(m)*ln [miR-140-5p]+(n)*ln [miR-223-5p]+(o)*ln [miR-582-3p]+(p)*ln [miR-122-3p]+(q)*ln [miR-589-5p]; wherein: (a)=any number from 23 to 28; (b)=any number from 7 to 11; (c)=any number from 1 to 6; (d)=any number from 3 to 8; (e)=any number from 6 to 11; (f)=any number from 5 to 11; (g)=any number from 1 to 5; (h)=any number from 0.5 to 4; (i)=any number from 5 to 11; (j)=any number from 6 to 13; (k)=any number from 3 to 8; (l)=any number from 0.1 to 2; (m)=any number from 1 to 4; (n)=any number from 1 to 5; (o)=any number from 0.5 to 4; (p)=any number from 0.1 to 3; and (q)=any number from 0.1 to 4; and wherein “[X]” refers to the amount of “X” in the biological sample, and Ln represents the natural log; wherein the human subject is identified as experiencing or at risk of experiencing an acute heart allograft rejection comprising an AMR if the AMR signature score is equal to or higher than about 65.

Some aspects of the present disclosure are directed to a method of identifying a human subject experiencing or at risk of experiencing an acute heart allograft rejection comprising an AMR following a heart transplant comprising: obtaining a biological sample from the human subject, wherein the biological sample comprises RNA; measuring the level of a panel of miRs in the biological sample, wherein the panel of miRs comprises miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, and miR-589-5p; and determining an AMR signature score according to the following formula: AMR signature score=222.41−(a)*ln [miR-23a-3p]−(b)*ln [miR-484]−(c)*ln [miR-340-5p]+(d)*ln [miR-193a-5p]−(e)*ln [miR-215-5p]+(f)*ln [miR-142-3p]−(g)*ln [miR-374a-5p]+(h)*ln [miR-1307]+(i)*ln [miR-185-3p]+(j)*ln [miR-4433b-3p]+(k)*ln [miR-130b-3p]+(1)*ln [miR-331-5p]+(m)*ln [miR-140-5p]+(n)*ln [miR-223-5p]+(o)*ln [miR-582-3p]+(p)*ln [miR-122-3p]+(q)*ln [miR-589-5p]; wherein: (a)=any number from 23 to 28; (b)=any number from 7 to 11; (c)=any number from 1 to 6; (d)=any number from 3 to 8; (e)=any number from 6 to 11; (f)=any number from 5 to 11; (g)=any number from 1 to 5; (h)=any number from 0.5 to 4; (i)=any number from 5 to 11; (j)=any number from 6 to 13; (k)=any number from 3 to 8; (l)=any number from 0.1 to 2; (m)=any number from 1 to 4; (n)=any number from 1 to 5; (o)=any number from 0.5 to 4; (p)=any number from 0.1 to 3; and (q)=any number from 0.1 to 4; and wherein “[X]” refers to the amount of “X” in the biological sample, and “Ln” represents the natural log; wherein the human subject is identified as experiencing or at risk of experiencing an acute heart allograft rejection comprising an AMR if the AMR signature score is equal to or higher than about 65.

Some aspects of the present disclosure are directed to a method of diagnosing an acute heart allograft rejection comprising an AMR after a heart transplant in a human subject, comprising obtaining a biological sample from the human subject, wherein the biological sample comprises RNA; measuring the level of a panel of miRs in the biological sample, wherein the panel of miRs comprises miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-3³¹-5p, miR-140-5p, miR-223-5p, miR-582-3p, miR-122-3p, and miR-589-5p; and determining an AMR signature score according to the following formula: 222.41−(a)*ln [miR-23a-3p]−(b)*ln [miR-484]−(c)*ln [miR-340-5p]+(d)*ln [miR-193a-5p]−(e)*ln [miR-215-5p]+(f)*ln [miR-142-3p]−(g)*ln [miR-374a-5p]+(h)*ln [miR-1307]+(i)*ln [miR-185-3p]+(j)*ln [miR-4433b-3p]+(k)*ln [miR-130b-3p]+(1)*ln [miR-331-5p]+(m)*ln [miR-140-5p]+(n)*ln [miR-223-5p]+(o)*ln [miR-582-3p]+(p)*ln [miR-122-3p]+(q)*ln [miR-589-5p]; wherein: (a)=any number from 23 to 28; (b)=any number from 7 to 11; (c)=any number from 1 to 6; (d)=any number from 3 to 8; (e)=any number from 6 to 11; (f)=any number from 5 to 11; (g)=any number from 1 to 5; (h)=any number from 0.5 to 4; (i)=any number from 5 to 11; (j)=any number from 6 to 13; (k)=any number from 3 to 8; (l)=any number from 0.1 to 2; (m)=any number from 1 to 4; (n)=any number from 1 to 5; (o)=any number from 0.5 to 4; (p)=any number from 0.1 to 3; and (q)=any number from 0.1 to 4; and wherein “[X]” refers to the amount of “X” in the biological sample, and “Ln” represents the natural log; wherein the human subject is identified as experiencing or at risk of experiencing an acute heart allograft rejection comprising an AMR if the AMR signature score is equal to or higher than about 65.

In some aspects, the method further comprises isolating RNA from the biological sample. In some aspects, the RNA comprises one or more miRs.

Some aspects of the present disclosure are directed to a method of identifying a human subject experiencing or at risk of experiencing an acute heart allograft rejection comprising an AMR following a heart transplant comprising measuring the level of a panel of miRs in a biological sample obtained from the human subject, wherein the panel of miRs comprises miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, and miR-589-5p; and determining an AMR signature score according to the following formula: AMR signature score=222.41−(a)*ln [miR-23a-3p]−(b)*ln [miR-484]−(c)*ln [miR-340-5p]+(d)*ln [miR-193a-5p]−(e)*ln [miR-215-5p]+(f)*ln [miR-142-3p]−(g)*ln [miR-374a-5p]+(h)*ln [miR-1307]+(i)*ln [miR-185-3p]+(j)*ln [miR-4433b-3p]+(k)*ln [miR-130b-3p]+(1)*ln [miR-331-5p]+(m)*ln [miR-140-5p]+(n)*ln [miR-223-5p]+(o)*ln [miR-582-3p]+(p)*ln [miR-122-3p]+(q)*ln [miR-589-5p]; wherein: (a)=any number from 23 to 28; (b)=any number from 7 to 11; (c)=any number from 1 to 6; (d)=any number from 3 to 8; (e)=any number from 6 to 11; (f)=any number from 5 to 11; (g)=any number from 1 to 5; (h)=any number from 0.5 to 4; (i)=any number from 5 to 11; (j)=any number from 6 to 13; (k)=any number from 3 to 8; (l)=any number from 0.1 to 2; (m)=any number from 1 to 4; (n)=any number from 1 to 5; (o)=any number from 0.5 to 4; (p)=any number from 0.1 to 3; and (q)=any number from 0.1 to 4; and wherein “[X]” refers to the level of “X” in the biological sample, and “Ln” represents the natural log; wherein the human subject is identified as experiencing or at risk of experiencing an acute heart allograft rejection comprising an AMR if the AMR signature score is equal to or higher than about 65.

Some aspects of the present disclosure are directed to a method of diagnosing an acute heart allograft rejection comprising an AMR after a heart transplant in a human subject, comprising measuring the level of a panel of miRs in a biological sample obtained from the human subject, wherein the panel of miRs comprises miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, and miR-589-5p; and determining an AMR signature score according to the following formula: AMR signature score=222.41−(a)*ln [miR-23a-3p]−(b)*ln [miR-484]−(c)*ln [miR-340-5p]+(d)*ln [miR-193a-5p]−(e)*ln [miR-215-5p]+(f)*ln [miR-142-3p]−(g)*ln [miR-374a-5p]+(h)*ln [miR-1307]+(i)*ln [miR-185-3p]+(j)*ln [miR-4433b-3p]+(k)*ln [miR-130b-3p]+(1)*ln [miR-331-5p]+(m)*ln [miR-140-5p]+(n)*ln [miR-223-5p]+(o)*ln [miR-582-3p]+(p)*ln [miR-122-3p]+(q)*ln [miR-589-5p]; wherein: (a)=any number from 23 to 28; (b)=any number from 7 to 11; (c)=any number from 1 to 6; (d)=any number from 3 to 8; (e)=any number from 6 to 11; (f)=any number from 5 to 11; (g)=any number from 1 to 5; (h)=any number from 0.5 to 4; (i)=any number from 5 to 11; (j)=any number from 6 to 13; (k)=any number from 3 to 8; (l)=any number from 0.1 to 2; (m)=any number from 1 to 4; (n)=any number from 1 to 5; (o)=any number from 0.5 to 4; (p)=any number from 0.1 to 3; and (q)=any number from 0.1 to 4; and wherein “[X]” refers to the leel of “X” in the biological sample, and “Ln” represents the natural log; wherein acute heart allograft rejection comprising an AMR is diagnosed if the AMR signature score is equal to or higher than about 65.

Some aspects of the present disclosure are directed to a method of treating an acute heart allograft rejection in a human subject in need thereof, comprising measuring the level of a panel of miRs in a biological sample obtained from the human subject, wherein the panel of miRs comprises miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, and miR-589-5p; determining an AMR signature score according to the following formula: AMR signature score=222.41−(a)*ln [miR-23a-3p]−(b)*ln [miR-484]−(c)*ln [miR-340-5p]+(d)*ln [miR-193a-5p]−(e)*ln [miR-215-5p]+(f)*ln [miR-142-3p]−(g)*ln [miR-374a-5p]+(h)*ln [miR-1307]+(i)*ln [miR-185-3p]+(j)*ln [miR-4433b-3p]+(k)*ln [miR-130b-3p]+(1)*ln [miR-331-5p]+(m)*ln [miR-140-5p]+(n)*ln [miR-223-5p]+(o)*ln [miR-582-3p]+(p)*ln [miR-122-3p]+(q)*ln [miR-589-5p]; wherein: (a)=any number from 23 to 28; (b)=any number from 7 to 11; (c)=any number from 1 to 6; (d)=any number from 3 to 8; (e)=any number from 6 to 11; (f)=any number from 5 to 11; (g)=any number from 1 to 5; (h)=any number from 0.5 to 4; (i)=any number from 5 to 11; (j)=any number from 6 to 13; (k)=any number from 3 to 8; (l)=any number from 0.1 to 2; (m)=any number from 1 to 4; (n)=any number from 1 to 5; (o)=any number from 0.5 to 4; (p)=any number from 0.1 to 3; and (q)=any number from 0.1 to 4; and administering an immunosuppressive therapy to the human subject identified as having an AMR signature score is equal to or higher than about 65; wherein “[X]” refers to the level of “X” in the biological sample.

In some aspects, (a)=any number from 24 to 27; (b)=any number from 8.5 to 10.5; (c)=any number from 2 to 4; (d)=any number from 4.5 to 6.5; (e)=any number from 7.5 to 9.5; (f)=any number from 7 to 9; (g)=any number from 2 to 4; (h)=any number from 1.0 to 1.75; (i)=any number from 7 to 9; (j)=any number from 9 to 11; (k)=any number from 5 to 7; (l)=any number from 1 to 2; (m)=any number from 1.5 to 2.5; (n)=any number from 1.5 to 2.5; (o)=any number from 0.7 to 1.7; (p)=any number from 0.5 to 1.5; and (q)=any number from 1.4 to 2.4.

In some aspects, (a)=about 25.44; (b)=about 9.33; (c)=about 3.39; (d)=about 5.82; (e)=about 8.24; (f)=about 8.62; (g)=about 2.75; (h)=about 1.43; (i)=about 7.95; (j)=about 9.69; (k)=about 5.47; (l)=about 0.60; (m)=about 2.05; (n)=about 2.24; (o)=about 1.40; (p)=about 0.87; and (q)=about 1.69. In some aspects, the miR panel further comprises one or more additional miR.

In some aspects, the method further comprises administering to the human subject an immunosuppressive therapy. In some aspects, the immunosuppressive therapy comprises administering a therapy selected from the group consisting of intravenous immunoglobulin, plasmapheresis, bortezomib, carfilzomib, rituximab, eculizumab, a corticosteroid, an anti-thymocyte globulin, tacrolimus, cyclosporine, sirolimus, everolimus, myocophenolate mofeitil, azathioprine, tocilizumab, belatacept, and any combination thereof.

In some aspects, the biological sample is a blood-based sample comprising whole blood, serum, plasma, or any combination thereof. In some aspects, the level of the panel of miRs is measured using small RNA/microRNA/RNA sequencing, array cards, microarray hybridization, probe-based assay, a northern blot, isothermal nucleic acid amplification (iNAAT), CRISPR, quantitative reverse-transcriptase PCR (qRT-PCR) or real time PCR (RT-PCR), or any combination thereof. In some aspects, the level of the panel of miRs is measured using a microfluidic array comprising one or more RNA-hybridization probes.

Some aspects of the present disclosure are directed to a kit comprising: one or more RNA-hybridization probes, wherein at least one of the RNA-hybridization probes hybridizes to an miR selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326, and any combination thereof, and instructions for measuring the level of a panel of miRs according to a method disclosed herein.

Some aspects of the present disclosure are directed to a kit comprising: one or more RNA-hybridization probes, wherein at least one of the RNA-hybridization probes hybridizes to an miR selected from the group consisting of miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, miR-589-5p, and any combination thereof, and instructions for measuring the level of a panel of miRs according to a method disclosed herein.

Some aspects of the present disclosure are directed to a kit comprising: an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326; and instructions for measuring the level of a panel of miRs according to a method disclosed herein.

Some aspects of the present disclosure are directed to a kit comprising: an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p; and instructions for measuring the level of a panel of miRs according to a method disclosed herein.

Some aspects of the present disclosure are directed to a method for generating a microRNA panel for detecting a transplant rejection, the method comprising: obtaining, by a processor, a plurality of sequence reads of microRNAs for one or more samples obtained from one or more subjects; filtering, by the processor, the plurality of sequence reads of microRNAs by removing one or more sequence reads of microRNAs from the plurality of sequence reads of microRNAs based on predetermined criteria; identifying, by the processor, nucleic acid targets from a filtered plurality of sequence reads of microRNAs; identifying, by the processor, a set of microRNAs in the filtered plurality of sequence reads of microRNAs based on the nucleic acid targets; identifying, by the processor, differentially expressed microRNAs from the set of miRs in Acute Cellular Rejection (ACR) or antibody-mediated rejection (AMR); generating, by the processor, a first logistic regression model using the differentially expressed microRNAs; and identifying, by the processor, one or more microRNAs from the differentially expressed microRNAs based on the first logistic regression model, wherein the one or more microRNAs are used to diagnose ACR if the differentially expressed microRNAs correspond to ACR or the one or more microRNAs are used to diagnose AMR if the differentially expressed microRNAs correspond to AMR.

In some aspects, the method further comprises: generating, by the processor, a second logistic regression model using microRNA expression data of each of the one or more samples corresponding to the one or more microRNAs; and identifying, by the processor, a first threshold score indicating a likelihood of ACR or a second threshold score indicating a likelihood of AMR based on the second logistic regression model. In some aspects, the method further comprises: receiving, by the processor, a request to determine whether a patient is diagnosed with ACR or AMR using a patient sample, wherein the patient is a heart transplant recipient; obtaining, by the processor, a plurality of patient sequence reads of microRNAs for the patient sample; identifying, by the processor, patient microRNA expression data from the plurality of patient sequence reads of microRNAs corresponding to the one or more microRNAs; and plotting, by the processor, the patient microRNA expression data on the second logistic regression model; generating, by the processor, an ACR score or an AMR score for the patient based on the patient microRNA expression data, as plotted on the second logistic regression model; determining, by the processor, that the patient is diagnosed with ACR if the ACR score is equal to or greater than the first threshold score or diagnosed with AMR if the AMR score is greater than or equal to the second threshold score; and identifying, by the processor, a heart allograft rejection in the patient based on the patient being diagnosed with ACR or AMR. In some aspects, the method further comprises identifying, by the processor, a treatment for the patient based on determining the patient is diagnosed with ACR or AMR. In some aspects, the nucleic acid targets are implicated in ACR or AMR.

In some aspects, the plurality of sequence reads are obtained by sequencing one or more samples. In some aspects, the predetermined criteria includes removing the one or more sequence reads from the plurality of sequence reads that a 3′ adapter barcode sequences, random-barcode sequences, UniVec contaminants, or sequence reads <15 base pair. In some aspects, the method further comprises executing, by the processor, a principle component analysis on the plurality of sequence reads of microRNAs after removing the one or more sequence reads.

In some aspects, identifying the differentially expressed microRNAs comprises: adjusting, by the processor, the differentially expressed microRNAs for clinical covariates. In some aspects, the clinical covariates include age, sex, race, or body-mass index.

In some aspects, the first logistic regression model is fitted with a LASSO penalty. In some aspects, a tuning parameter of the LASSO penalty is selected by 10-fold cross-validation to minimize model deviance.

In some aspects, the differentially expressed microRNAs used to generate the first logistic regression model have an unadjusted probability value less than 0.10. In some aspects, the generating the first logistic regression model comprises: normalizing, by the processor, counts of the differentially expressed microRNAs; transforming, by the processor, the normalized counts logarithmically to generate log counts; and standardizing, by the processor, log counts for each respective differentially expressed microRNA to have a mean value of zero and a variance of 1.

Some aspects of the present disclosure are directed to a system for generating a microRNA panel for detecting a transplant rejection, the system comprising: a memory; and a processor coupled to the memory, the processor configured to: obtain a plurality of sequence reads of microRNAs for one or more samples obtained from one or more subjects; filter the plurality of sequence reads of microRNAs by removing one or more sequence reads of microRNAs from the plurality of sequence reads of microRNAs based on predetermined criteria; identify nucleic acid targets from a filtered plurality of sequence reads of microRNAs; identify a set of microRNAs in the filtered plurality of sequence reads of microRNAs based on the nucleic acid targets; identify differentially expressed microRNAs from the set of miRs in Acute Cellular Rejection (ACR) or antibody-mediated rejection (AMR); generate a first logistic regression model using the differentially expressed microRNAs; and identify one or more microRNAs from the differentially expressed microRNAs based on the first logistic regression model, wherein the one or more microRNAs are used to diagnose ACR if the differentially expressed microRNAs correspond to ACR or the one or more microRNAs are used to diagnose AMR if the differentially expressed microRNAs correspond to AMR.

In some aspects, the processor is further configured to: generate a second logistic regression model using microRNA expression data of each of the one or more samples corresponding to the one or more microRNAs; and identify a first threshold score indicating a likelihood of ACR or a second threshold score indicating a likelihood of AMR based on the second logistic regression model. In some aspects, the processor is further configured to: receive a request to determine whether a patient is diagnosed with ACR or AMR using a patient sample, wherein the patient is a heart transplant recipient; obtain a plurality of patient sequence reads of microRNAs for a patient sample; identify patient microRNA expression data from the plurality of patient sequence reads of microRNAs corresponding to the one or more microRNAs; and plot the patient microRNA expression data on the second logistic regression model; generate an ACR score or an AMR score for the patient based on the patient microRNA expression data, as plotted on the second logistic regression model; determine that the patient is diagnosed with ACR if the ACR score is equal to or greater than the first threshold score or diagnosed with AMR if the AMR score is greater than or equal to the second threshold score; and identify a heart allograft rejection in the patient based on the patient being diagnosed with ACR or AMR. In some aspects, the processor is further configured to identify a treatment for the patient based on determining the patient is diagnosed with ACR or AMR. In some aspects, the nucleic acid targets are implicated in ACR or AMR.

In some aspects, obtaining the plurality of sequence reads of microRNAs by sequencing the one or more samples. In some aspects, the predetermined criteria includes removing the one or more sequence reads from the plurality of sequence reads that are 3′ adapter barcode sequences, random-barcode sequences, UniVec contaminants, or sequence reads <15 base pair. In some aspects, the processor is further configured to execute a principle component analysis on the plurality of sequence reads of microRNAs after removing the one or more sequence reads.

In some aspects, when identifying the differentially expressed microRNAs the processor is further configured to: adjust the differentially expressed microRNAs for clinical covariates. In some aspects, the clinical covariates include age, sex, race, or body-mass index.

In some aspects, the first logistic regression model is fitted with a LASSO penalty. In some aspects, the tuning parameter of the LASSO penalty is selected by 10-fold cross-validation to minimize model deviance.

In some aspects, the differentially expressed microRNAs used to generate the first logistic regression model, have an unadjusted probability value less than 0.10. In some aspects, when generating the first logistic regression model, the processor is further configured to: normalize counts of the differentially expressed microRNAs; transform the normalized counts logarithmically to generate log counts; and standardize log counts for each respective differentially expressed microRNA to have a mean value of zero and a variance of 1.

Some aspects of the present disclosure are directed to a non-transitory computer-readable medium having instructions stored thereon, execution of which, by one or more processors of a device, causes the one or more processors to perform operations comprising: obtaining a plurality of sequence reads of microRNAs for one or more samples obtained from one or more subjects; filtering the plurality of sequence reads of microRNAs by removing one or more sequence reads of microRNAs from the plurality of sequence reads of microRNAs based on predetermined criteria; identifying nucleic acid targets from a filtered plurality of sequence reads of microRNAs; identifying a set of microRNAs in the filtered plurality of sequence reads of microRNAs based on the nucleic acid targets; identifying differentially expressed microRNAs from the set of miRs in Acute Cellular Rejection (ACR) or antibody-mediated rejection (AMR); generating a first logistic regression model using the differentially expressed microRNAs; and identifying one or more microRNAs from the differentially expressed microRNAs based on the first logistic regression model, wherein the one or more microRNAs are used to diagnose ACR if the differentially expressed microRNAs correspond to ACR or the one or more microRNAs are used to diagnose AMR if the differentially expressed microRNAs correspond to AMR.

In some aspects, the operations further comprising: generating a second logistic regression model using microRNA expression data of each of the one or more samples corresponding to the one or more microRNAs; and identifying a first threshold score indicating a likelihood of ACR or a second threshold score indicating a likelihood of AMR based on the second logistic regression model. In some aspects, the operations further comprising: receiving a request to determine whether a patient is diagnosed with ACR or AMR using a patient sample, wherein the patient is a heart transplant recipient; obtaining a plurality of sequence reads of microRNAs for the patient sample; identifying patient microRNA expression data from the plurality of patient sequence reads corresponding to the one or more microRNAs; and plotting the patient microRNA expression data on the second logistic regression model; generating an ACR score or an AMR score for the patient based on the patient microRNA expression data, as plotted on the second logistic regression model; determining that the patient is diagnosed with ACR if the ACR score is equal to or greater than the first threshold score or diagnosed with AMR if the AMR score is greater than or equal to the second threshold score; and identifying a heart allograft rejection in the patient based on the patient being diagnosed with ACR or AMR. In some aspects, the operations further comprise identifying a treatment for the patient based on determining the patient is diagnosed with ACR or AMR. In some aspects, the nucleic acid targets are implicated in ACR or AMR.

In some aspects, obtaining the plurality of sequence reads of microRNAs by sequencing the one or more samples. In some aspects, the predetermined criteria includes removing the one or more sequence reads from the plurality of sequence reads based on remove 3′ adapter barcode sequences, random-barcode sequences, UniVec contaminants, or sequence reads <15 base pair. In some aspects, the operations further comprise executing a principle component analysis on the plurality of sequence reads of microRNAs after removing the one or more sequence reads.

In some aspects, when identifying the differentially expressed microRNAs the operations further comprise: adjusting the differentially expressed microRNAs for clinical covariates. In some aspects, the clinical covariates include age, sex, race, or body-mass index.

In some aspects, the first logistic regression model is fitted with a LASSO penalty. In some aspects, the tuning parameter of the LASSO penalty is selected by 10-fold cross-validation to minimize model deviance.

In some aspects, the differentially expressed microRNAs used to generate the first logistic regression model have an unadjusted probability value less than 0.10. In some aspects, when the generating the first logistic regression model the operations further comprising: normalizing counts of the differentially expressed microRNAs; transforming the normalized counts logarithmically to generate log counts; and standardizing log counts for each respective differentially expressed microRNA to have a mean value of zero and a variance of 1.

Some aspects of the present disclosure are directed to a method for identifying an allograft rejection in a patient with a heart transplant, the method comprising: receiving, by the processor, a request to detect Acute Cellular Rejection (ACR) or antibody-mediated rejection (AMR) in a patient sample, wherein the patient sample is from the patient with a heart transplant; obtaining, by the processor, a plurality of patient sequence reads of microRNAs for the patient sample; identifying, by the processor, patient microRNA expression data from the plurality of patient sequence reads of microRNAs corresponding to one or more microRNAs, wherein the one or more microRNAs are used to detect ACR or AMR; and plotting, by the processor, the patient microRNA expression data on the second logistic regression model; generating, by the processor, an ACR score or an AMR score for the patient by: identifying, by the processor, a coefficient based on patient microRNA expression data as plotted on the second logistic regression model; generating, by the processor, a weighted ACR score or weighted AMR score by multiplying the coefficient with each natural log-transformed microRNA of the one or more microRNAs; and scaling, by the processor, the weighted ACR score or weighted AMR score based on a numerical range; determining, by the processor, that patient is diagnosed with ACR if the ACR score is equal to or greater than a first threshold corresponding to ACR score or diagnosed with AMR if the AMR score is greater than or equal to a second threshold score corresponding to AMR; and identifying, by the processor, the allograft rejection in the patient based on the patient being diagnosed with ACR or AMR.

Some aspects of the present disclosure are directed to a system for identifying an allograft rejection in a patient, the system comprising: a memory; and a processor coupled to the memory, the processor configured to: receive a request to detect Acute Cellular Rejection (ACR) or antibody-mediated rejection (AMR) in a patient sample, wherein the patient sample is from the patient with a heart transplant; obtain a plurality of patient sequence reads of microRNAs for the patient sample; identify patient microRNA expression data from the plurality of patient sequence reads of microRNAs corresponding to one or more microRNAs, wherein the one or more microRNAs are used to detect ACR or AMR; and plot the patient microRNA expression data on the second logistic regression model; generate an ACR score or an AMR score for the patient by: identify a coefficient based on patient microRNA expression data as plotted on the second logistic regression model; generate a weighted ACR score or weighted AMR score by multiplying the coefficient with each natural log-transformed microRNA of the one or more microRNAs; and scale the weighted ACR score or weighted AMR score based on a numerical range; determine that patient is diagnosed with ACR if the ACR score is equal to or greater than a first threshold corresponding to ACR score or diagnosed with AMR if the AMR score is greater than or equal to a second threshold score corresponding to AMR; identify the allograft rejection in the patient based on the patient being diagnosed with ACR or AMR.

Some aspects of the present disclosure are directed to a non-transitory computer-readable medium having instructions stored thereon, execution of which, by one or more processors of a device, cause the one or more processors to perform operations comprising: receiving a request to detect Acute Cellular Rejection (ACR) or antibody-mediated rejection (AMR) in a patient sample, wherein the patient sample is from the patient with a heart transplant; obtaining a plurality of patient sequence reads of microRNAs for the patient sample; identifying patient microRNA expression data from the plurality of patient sequence reads of microRNAs corresponding to one or more microRNAs, wherein the one or more microRNAs are used to detect ACR and AMR; and plotting the patient microRNA expression data on the second logistic regression model; generating an ACR score and an AMR score for the patient by: identifying a coefficient based on patient microRNA expression data as plotted on the second logistic regression model; generating a weighted ACR score or weighted AMR score by multiplying the coefficient with each natural log-transformed microRNA of the one or more microRNAs; and scaling the weighted ACR score or weighted AMR score based on a numerical range; determining that the patient is diagnosed with ACR if the ACR score is equal to or greater than a first threshold corresponding to ACR score or diagnosed with AMR if the AMR score is greater than or equal to a second threshold score corresponding to AMR; and identifying an allograft rejection in the patient based on the patient being diagnosed with ACR or AMR.

Some aspects of the present disclosure are directed to a method for determining a risk of an allograft rejection in a patient with a heart transplant, the method comprising: receiving, by the processor, a request to detect Acute Cellular Rejection (ACR) or antibody-mediated rejection (AMR) in a patient sample, wherein the patient sample is from the patient with a heart transplant; obtaining, by the processor, a plurality of patient sequence reads of microRNAs for the patient sample; identifying, by the processor, patient microRNA expression data from the plurality of patient sequence reads of microRNAs corresponding to one or more microRNAs, wherein the one or more microRNAs are used to detect ACR or AMR; and plotting, by the processor, the patient microRNA expression data on the second logistic regression model; generating, by the processor, an ACR score or an AMR score for the patient by: identifying, by the processor, a coefficient based on patient microRNA expression data as plotted on the second logistic regression model; generating, by the processor, a weighted ACR score or weighted AMR score by multiplying the coefficient with each natural log-transformed microRNA of the one or more microRNAs; and scaling, by the processor, the weighted ACR score or weighted AMR score based on a numerical range; and determining, by the processor, that patient is at risk of being diagnosed with ACR if the ACR score is equal to or greater than a first threshold corresponding to ACR score or at risk of being diagnosed with AMR if the AMR score is greater than or equal to a second threshold score corresponding to AMR.

Some aspects of the present disclosure are directed to a system for identifying an allograft rejection in a patient, the system comprising: a memory; and a processor coupled to the memory, the processor configured to: receive a request to detect Acute Cellular Rejection (ACR) or antibody-mediated rejection (AMR) in a patient sample, wherein the patient sample is from the patient with a heart transplant; obtain a plurality of patient sequence reads of microRNAs for the patient sample; identify patient microRNA expression data from the plurality of patient sequence reads of microRNAs corresponding to one or more microRNAs, wherein the one or more microRNAs are used to detect ACR or AMR; and plot the patient microRNA expression data on the second logistic regression model; generate an ACR score or an AMR score for the patient by: identifying a coefficient based on patient microRNA expression data as plotted on the second logistic regression model; generating a weighted ACR score or weighted AMR score by multiplying the coefficient with each natural log-transformed microRNA of the one or more microRNAs; and scaling the weighted ACR score or weighted AMR score based on a numerical range; and determine that patient is at risk of being diagnosed with ACR if the ACR score is equal to or greater than a first threshold corresponding to ACR score or at risk of being diagnosed with AMR if the AMR score is greater than or equal to a second threshold score corresponding to AMR.

Some aspects of the present disclosure are directed to a non-transitory computer-readable medium having instructions stored thereon, execution of which, by one or more processors of a device, cause the one or more processors to perform operations comprising: receiving a request to detect Acute Cellular Rejection (ACR) or antibody-mediated rejection (AMR) in a patient sample, wherein the patient sample is from the patient with a heart transplant; obtaining a plurality of patient sequence reads of microRNAs for the patient sample; identifying patient microRNA expression data from the plurality of patient sequence reads of microRNAs corresponding to one or more microRNAs, wherein the one or more microRNAs are used to detect ACR and AMR; and plotting the patient microRNA expression data on the second logistic regression model; generating an ACR score and an AMR score for the patient by: identifying a coefficient based on patient microRNA expression data as plotted on the second logistic regression model; generating a weighted ACR score or weighted AMR score by multiplying the coefficient with each natural log-transformed microRNA of the one or more microRNAs; and scaling the weighted ACR score or weighted AMR score based on a numerical range; and determining, by the processor, that patient is at risk of being diagnosed with ACR if the ACR score is equal to or greater than a first threshold corresponding to ACR score or at risk of being diagnosed with AMR if the AMR score is greater than or equal to a second threshold score corresponding to AMR.

### BRIEF DESCRIPTION OF THE DRAWINGS

The accompanying drawings, which are incorporated herein and form part of the specification, illustrate the present disclosure and, together with the description, further serve to explain the principles of the disclosure and enable a person skilled in the relevant art to make and use the disclosure.

FIG. 1A is a schematic representation of a sample study overview according to an aspect of the present disclosure. Blood samples were collected in transplant patients just prior to a scheduled or clinically indicated endomyocardial biopsy. Biopsy samples were reviewed by the institutional pathologist to grade the presence/severity of ACR and AMR. Plasma was isolated from whole blood. The small RNA transcriptome was then extracted, isolated, and sequenced on a next generation sequencer using shotgun sequencing. Sequence data was aligned to the human genome and miRBase, and the microRNA (miR) transcriptome was then determined for each sample. Biostatistical tools were then used to distinguish the miR signatures of ACR and AMR.

FIGS. 1B-1C are volcano plots showing the differential expression of various miRs in ACR (FIG. 1B) and AMR (FIG. 1C) samples. The x-axis line represents log fold +/−0.5. The y-axis line represents unadjusted p=0.05. miRs with a log-fold change ≥0.5, but p-value <0.05 are present in the regions designated by IV and VI; miRs with a p-value >0.05, but log-fold change <±0.5 are present in the region designated by II; differentially expressed miRs with a log-fold change ≥±0.5 and p-value <0.05 are present in the regions designated by I and III (FIGS. 1B-1C).

FIGS. 1D-1K are box plots illustrating miR levels (log-transformed microRNA reads per million) for four differentially expressed miRs for ACR (FIGS. 1D-1G) and AMR (FIGS. 1H-1K), compared between controls and patients at three time points (pre-rejection, during rejection and post-rejection). Pre- and post-rejection were within 3 months of the rejection episode.

FIGS. 2A and 2C are graphical representations of the area under curve (AUC) for the receiver operator characteristics curve for miR panels diagnostic of ACR, and AMR, which are further summarized in FIGS. 2B and 2D, respectively. The miRs were selected to maximize diagnostic performance using LASSO regression. Test performance characteristics were reported for blood samples collected from 8 days to 2.6 years after transplant.

FIG. 3 is a block diagram of a system for detecting an allograft rejection based on miRs, according to some aspects.

FIG. 4 is a flowchart illustrating a process of generating a miR panel, according to some aspects.

FIG. 5 is a flowchart illustrating a process for identifying a heart allograft rejection in a patient, according to some aspects.

FIG. 6 illustrates a clinical application of ACR and AMR miR scores.

FIG. 7 is a block diagram of example components of a device according to an aspect.

The drawing in which an element first appears is typically indicated by the leftmost digit or digits in the corresponding reference number. In the drawings, like reference numbers may indicate identical or functionally similar elements.

### DETAILED DESCRIPTION

Some aspects of the present disclosure are directed to microfluidic arrays, chips (e.g., microfluidic chips), and panels comprising one or more RNA-hybridization probes, wherein at least one of the RNA-hybridization probes hybridizes to an miR selected from the group consisting of miR-223-3p, miR-361-3p, miR-3615, miR-24-3p, miR-182-5p, miR-374a-5p, miR-23a-3p, miR-30e-5p, miR-582-3p, miR-130b-3p, miR-326 92, miR-1299, miR-23a-3p, miR-145-5p, miR-1249-3p, miR-27a-3p, miR-215-5p, miR-145-3p, miR-10b-5p, miR-582-3p, let-7b-3p, miR-142-3p, miR-450b-5p, miR-140-5p, miR-374a-5p, miR-17-5p, miR-143-3p, miR-130b-3p, miR-1-3p, miR-542-3p, miR-484, miR-345-5p, miR-125a-5p, miR-338-5p, miR-769-5p, miR-193a-5p, miR-454-3p, miR-223-5p, and let-7d-3p. Some aspects of the present disclosure are directed to a chip (e.g., a microfluidic chip). In some aspects, the microfluidic arrays, chips (e.g., microfluidic chips), and panels are used in a method of identifying a subject experiencing or at risk of experiencing an acute heart allograft rejection. In some aspects, the acute allograft rejection comprises ACR. In some aspects, the acute allograft rejection comprises AMR.

Other aspects of the present disclosure are directed to methods of identifying a human subject experiencing or at risk of experiencing an acute heart allograft rejection by measuring the expression of a panel of miRs in a biological sample obtained from the subject.

Other aspects of the present disclosure are directed to methods of identifying miRs that are differentially expressed in patients experiencing or at risk of experiencing an organ rejection. Provided herein are system, apparatus, device, method, and/or computer program product aspects, and/or combinations and sub-combinations thereof, for detecting an allograft rejection based on miRs. According to various aspects, a model for developing a miR panel indicative of allograft rejection is generated. The model is then applied to a patient sample to indicate a status or likelihood of allograft rejection for the patient.

### I. Terms

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth by a variance of 10 percent, up or down (higher or lower).

As used herein, the term “acute heart allograft rejection” refers to the condition arising in heart transplant patients wherein the transplanted heart is rejected by the patient. As used herein, “acute cellular rejection” or “ACR” refers to a cellular-based acute heart allograft rejection wherein the transplant recipient's immune system, e.g., T cells, target and attack the donor heart. ACR presents as a mononuclear inflammatory response infiltrating myocardial tissue with predominant lymphocytic cells. Traditional methods of immunohistologic assessment can confirm the presence of CD-4 and CD-8 positive T lymphocytes with high affinity to interleukin-2 receptors. Presence of increased intercellular adhesion molecules with high MHC-II expression on cardiac myocytes is also observed. As used herein, “antibody-mediated rejection,” or “acute humoral/antibody rejection,” or “AMR” refers to a humoral-based acute heart allograft rejection wherein recipient antibodies react to donor antigens present on the transplanted heart, leading to deposition of immunoglobulins and complements within the myocardial capillary bed. AMR leads to intravascular macrophage accumulation with interstitial edema, hemorrhage and neutrophilic infiltration in and around capillaries.

As used herein, the term “afflicted with” when used in conjunction with a particular disease or condition refers to a subject that currently has or is currently suffering from a particular disease or condition. For example, a subject afflicted with an ACR is a subject that currently is experiencing the signs or symptoms of ACR, e.g., the subject is currently experiencing an immune response against a donor heart. Conversely, the term “at risk of developing” refers to a subject who has not yet started experiencing the symptoms and/or effects of a particular disease, but which subject may be predisposed or likely to develop the symptoms and/or effects of the disease in the near future (e.g., within the next 1, 2, 3, 4, 5, 6, 7, 8, 9, 10, 11, 12, or more weeks). A subject “at risk” of developing a disease may be experiencing the signs of the disease, e.g., increased accumulation of immunoglobulins in an AMR, without yet experiencing or presenting with any of the symptoms, e.g., interstitial edema, hemorrhage and neutrophilic infiltration in and around capillaries for AMR.

In some aspects, the methods and/or arrays described herein are used in the “surveillance” of a subject following an organ transplantation (e.g., a heart transplantation). “Surveillance,” as used herein, refers to the ongoing monitoring of a patient following organ transplantation to see if the patient has developed a rejection (e.g., AMR or ACR). A patient under surveillance need not have any signs or symptoms of rejection.

In some aspects, the methods and/or arrays described herein are used in the “diagnosis” of an organ transplantation rejection (e.g., AMR or ACR) in a subject. “Diagnosis,” as used herein, refers to the identification of a patient experiencing an organ transplantation rejection (e.g., AMR or ACR), wherein the patient is experiencing one or more signs or symptoms of rejection prior to or concurrently with the diagnosis.

In some aspects, the methods and/or arrays described herein are used in the “prediction” of an organ transplantation rejection (e.g., AMR or ACR). As used herein, “prediction” refers to identifying a patient that is at risk of developing a rejection (e.g., AMR or ACR), wherein the patient is not experiencing any signs or symptoms of rejection.

An “array” (sometimes referenced as a “microarray”), as used herein, refers to two or three dimensional arrangement of addressable regions bearing a particular chemical moiety or moieties (called “probes” or “probe molecules”) (e.g., RNA-hybridization probes) associated with that region. An array is “addressable” in that it has multiple regions of different moieties (e.g., RNA-hybridization probes) such that a region (an “array feature” or “spot” of the array) at a particular predetermined location (an “address”) on the array will detect a particular target or class of targets (although an array feature may incidentally detect non-targets of that array feature). In the case of an array, the “target” will be referenced as a moiety in a mobile phase (typically fluid), to be detected by probes (sometimes referenced as “target probes”) which are bound to the substrate at the various regions. The probes may be bound to the substrate by interactions that include, for example, covalent and/or electrostatic interactions. In relation to the array, the mobile phase comprises or is prepared from the biological sample, and comprises the target, i.e., the miRs. “Interrogating” the array refers to obtaining information from the array, especially information about targets binding to the array. In some aspects, the array comprises a fluidic chip. In some aspects, the array comprises a pneumatic system, comprising one or more mobile pins and one or more chambers. In some aspects, the array comprises a capillary system, wherein capillary forces move fluid through the array.

As used herein, the term “biological sample” refers to a sample obtained from a subject. In some aspects, the sample is a “blood-based sample,” which refers to a sample obtained from a subject that includes blood or a component of blood. A blood-based sample can comprise whole blood. In some aspects, the biological sample comprises or consists essentially of plasma. In some aspects, a biological sample comprises a tissue biopsy obtained from the subject. In some aspects, the tissue biopsy is obtained from the organ transplant tissue. The methods disclosed herein for identifying miRs that may be indicative of organ rejection can be applied to any organ transplant. As such, the biological sample can depend on the organ that has been transplanted. In some aspects, the biological sample comprises urine (e.g., for a kidney transplant). In some aspects, the biological sample comprises a bronchoalveolar lavage (e.g., for a lung transplant).

As used herein, the term “microfluidic” refers to a component or system that has microfluidic features, e.g., channels and/or chambers, which are generally fabricated on the micron or submicron scale. In some aspects, the channels or chambers have at least one cross-sectional dimension in the range of about 0.1 microns to about 1500 microns, more typically in the range of about 0.2 microns to about 1000 microns, still more typically in the range of about 0.4 microns to about 500 microns. Individual microfluidic features typically hold very small quantities of fluid, e.g., from about 10 nanoliters to about 5 milliliters, from about 100 nanoliters to about 2 milliliters, from about 200 nanoliters to about 500 microliters, or from about 500 nanoliters to about 200 microliters. An integrated microfluidic array device includes an array component joined to a microfluidic component, wherein the microfluidic component and the array component are in operable association with each other such that an array substrate of the array component is in fluid communication with a microfluidic feature of the microfluidic component. A microfluidic component is a component that includes a microfluidic feature and is adapted to being in operable association with an array component. An array component is a component that includes an array substrate and is adapted to being in operable association with a microfluidic component.

RNA, or ribonucleic acid, is a polymeric molecule with multiple biological roles. As used herein, the term “microRNA,” “miRNA,” or “miR” are used interchangeably to refer to a small single-stranded non-coding RNA molecule. In some aspects, a miR is about 15 to about 30 nucleotides in length (e.g., about 15, about 16, about 17, about 18, about 19, about 20, about 21, about 22, about 23, about 24, about 25, about 26, about 27, about 28, about 29, or about 30 nucleotides in length). miRs can have various natural functions, including RNA silencing and post-transcriptional regulation of gene expression. Structurally, a miR is a single stranded molecule that folds back over itself to form one or more hairpin loop structures.

An “RNA-hybridization probe,” as used herein, refers to an RNA molecule for detecting a target RNA molecule, wherein the RNA-hybridization probe has a specific sequence that is complementary to all or a portion of the target molecule. RNA-hybridization probes further comprise a detectable element, such as a fluorescent or radioactive label. In some aspects, the RNA-hybridization probe is further modified to increase stability.

The terms “subject” and “patient” are used interchangeably herein, and each refers to a human.

Various aspects described herein are described in further detail in the following subsections.

### II. Arrays of the Disclosure

Some aspects of the present disclosure are directed to devices comprising one or more RNA-hybridization probes, wherein the one or more RNA-hybridization probes hybridize to miRs disclosed herein. In some aspects, the device is a microfluidic array. In some aspects, the device is a chip (e.g., a microfluidic chip). The arrays described in the present disclosure comprise RNA-hybridization probes that hybridize to specific miRs that are identified herein as differentially expressed in subjects experiencing or at risk of experiencing ACR or AMR. As such, the arrays disclosed herein represent novel tools in not only identifying subjects experiencing or at risk of experiencing an acute heart allograft rejection, but that allow a clinician to readily differentiate between an ACR and an AMR.

Some aspects of the present disclosure are directed to a microfluidic array comprising one or more RNA-hybridization probes, wherein at least one of the RNA-hybridization probes hybridizes to an miR selected from the group consisting of miR-223-3p, miR-361-3p, miR-3615, miR-24-3p, miR-182-5p, miR-374a-5p, miR-23a-3p, miR-30e-5p, miR-582-3p, miR-130b-3p, miR-326 92, miR-1299, miR-23a-3p, miR-145-5p, miR-1249-3p, miR-27a-3p, miR-215-5p, miR-145-3p, miR-10b-5p, miR-582-3p, let-7b-3p, miR-142-3p, miR-450b-5p, miR-140-5p, miR-374a-5p, miR-17-5p, miR-143-3p, miR-130b-3p, miR-1-3p, miR-542-3p, miR-484, miR-345-5p, miR-125a-5p, miR-338-5p, miR-769-5p, miR-193a-5p, miR-454-3p, miR-223-5p, and let-7d-3p.

In some aspects, the microfluidic array comprises one or more RNA-hybridization probes, wherein at least one of the RNA-hybridization probes hybridizes to an miR selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326, and any combination thereof.

In some aspects, the microfluidic array comprises at least two, at least three, at least four, at least five, at least six, at least seven, at least eight, at least nine, at least ten, or at least eleven RNA-hybridization probes selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326. In some aspects, the microfluidic array comprises a plurality of RNA-hybridization probes comprising a RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, an RNA-hybridization probe that hybridizes to miR-326. In some aspects, the microfluidic array comprises a plurality of RNA-hybridization probes consisting of an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, an RNA-hybridization probe that hybridizes to miR-326.

Some aspects of the present disclosure are directed to a microfluidic array for identifying a subject experiencing or at risk of developing an ACR, wherein the microfluidic array comprises a plurality of RNA-hybridization probes comprising an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326. In some aspects, the microfluidic array comprises a plurality of RNA-hybridization probes consisting of an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326.

Some aspects of the present disclosure are directed to a microfluidic array comprising one or more RNA-hybridization probes, wherein at least one of the RNA-hybridization probes hybridizes to an miR selected from the group consisting of miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, miR-589-5p, and any combination thereof. In some aspects, the microfluidic array comprises at least two, at least three, at least four, at least five, at least six, at least seven, at least eight, at least nine, at least ten, at least eleven, at least twelve, at least thirteen, at least fourteen, at least fifteen, or at least sixteen RNA-hybridization probes selected from the group consisting of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p.

In some aspects, the microfluidic array comprises a plurality of RNA-hybridization probes comprising an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p. In some aspects, the plurality of RNA-hybridization probes consists of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p.

Some aspects of the present disclosure are directed to a microfluidic array for identifying a subject experiencing or at risk of developing an AMR, wherein the microfluidic array comprises a plurality of RNA-hybridization probes comprising an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p. In some aspects, the plurality of RNA-hybridization probes consists of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p.

### III. RNA-Hybridization Probe Panels

Some aspects of the present disclosure are directed to a panel of RNA-hybridization probes, which is capable of being interrogated to identify a subject experiencing or at risk of experiencing acute heart allograft rejection, e.g., an ACR or an AMR. In some aspects, the panel of RNA-hybridization probes hybridize to one or more miR selected from the group consisting of miR-223-3p, miR-361-3p, miR-3615, miR-24-3p, miR-182-5p, miR-374a-5p, miR-23a-3p, miR-30e-5p, miR-582-3p, miR-130b-3p, miR-326 92, miR-1299, miR-23a-3p, miR-145-5p, miR-1249-3p, miR-27a-3p, miR-215-5p, miR-145-3p, miR-10b-5p, miR-582-3p, let-7b-3p, miR-142-3p, miR-450b-5p, miR-140-5p, miR-374a-5p, miR-17-5p, miR-143-3p, miR-130b-3p, miR-1-3p, miR-542-3p, miR-484, miR-345-5p, miR-125a-5p, miR-338-5p, miR-769-5p, miR-193a-5p, miR-454-3p, miR-223-5p, let-7d-3p, and any combination thereof, wherein the panel of RNA-hybridization probes is for use in identifying a human subject experiencing or at risk of developing an acute heart allograft rejection following a heart transplant.

In some aspects, the panel of RNA-hybridization probes comprises at least two, at least three, at least four, at least five, at least six, at least seven, at least eight, at least nine, at least ten, or at least eleven RNA-hybridization probes selected from the group consisting of an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, an RNA-hybridization probe that hybridizes to miR-326, and any combination thereof, wherein the panel of RNA-hybridization probes is for use in identifying a human subject experiencing or at risk of developing ACR.

In some aspects, the panel of RNA-hybridization probes comprises an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326. In some aspects, the panel of RNA-hybridization probes is for use in identifying a human subject experiencing with or at risk of developing ACR.

In some aspects, the panel of RNA-hybridization probes consists of an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326. In some aspects, the panel of RNA-hybridization probes is for use in identifying a human subject experiencing with or at risk of developing ACR.

In some aspects, the panel of RNA-hybridization probes comprises at least two, at least three, at least four, at least five, at least six, at least seven, at least eight, at least nine, at least ten, at least eleven, at least twelve, at least thirteen, at least fourteen, at least fifteen, or at least sixteen RNA-hybridization probes selected from the group consisting of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p.

In some aspects, the panel of RNA-hybridization probes comprises an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p. In some aspects, the panel of RNA-hybridization probes is for use in identifying a human subject experiencing with or at risk of developing AMR.

In some aspects, the panel of RNA-hybridization probes consists of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to and miR-589-5p. In some aspects, the panel of RNA-hybridization probes is for use in identifying a human subject experiencing with or at risk of developing AMR.

### IV. Methods of the Disclosure

Described herein are examples of miRs that are differentially expressed in a human subject experiencing or at a risk of experiencing ACR or AMR following a heart transplant. As such, some aspects of the present disclosure are directed to methods of identifying a human subject experiencing or at risk of experiencing an acute heart allograft rejection by measuring the expression of one or miRs disclosed herein.

Further, the methods used to generate the panels of miR disclosed herein are readily adaptable to identifying differentially expressed miR associated with other types of organ transplant rejection. As such, some aspects of the present disclosure are directed to methods of identifying miRs that are differentially expressed in subjects experiencing or at risk of experiencing organ transplant rejection.

### IV.A. Methods of Identification

Some aspects of the present disclosure are directed to methods of identifying or diagnosing a subject as experiencing or at risk of experiencing an acute heart allograft rejection by measuring the expression of one or more miRs disclosed herein in a biological sample obtained from the subject. In some aspects, the acute heart allograft rejection comprises ACR. In some aspects, the acute heart allograft rejection comprises AMR.

In some aspects, the method comprises obtaining a biological sample from the subject. In some aspects, the biological sample is a blood-based sample. In some aspects, the blood-based sample comprises whole blood. In some aspects, the biological sample is a whole blood sample. In some aspects, the blood-based sample comprises serum. In some aspects, the biological sample is a serum sample. In some aspects, the blood-based sample comprises plasma. In some aspects, the biological sample is a plasma sample. In some aspects, the biological sample comprises a tissue biopsy obtained from organ transplant tissue. A sample suitable for the methods disclosed herein will comprise miRs. In some aspects, RNA is isolated from the biological sample. In some aspects, total RNA is isolated from the biological sample. In some aspects, small RNA, e.g., non-coding RNA, is isolated from the biological sample. In some aspects, miRs are isolated from the biological sample.

In some aspects of the present disclosure, the methods comprise measuring the level of a panel of miRs in the biological sample by contacting the biological sample with a microfluidic array disclosed herein. In some aspects, a whole blood sample is applied to the microfluidic array. In some aspects, a plasma sample is applied to the microfluidic array. In some aspects, a serum sample is applied to the microfluidic array. In some aspects, total RNA isolated from a blood-based biological sample is applied to the microfluidic array. In some aspects, small RNA isolated from a blood-based biological sample is applied to the microfluidic array. In some aspects, miRs isolated from a blood-based biological sample is applied to the microfluidic array.

In some aspects, the level of the particular miR is assayed, and a relative amount of the miR in the sample is determined.

### IV.A.1. ACR

In some aspects, the method comprises identifying or diagnosing a subject as experiencing or at risk of experiencing an ACR, wherein the method comprises measuring the level of a panel of miRs in a biological sample obtained from the subject, wherein the panel of miRs comprises miR-30e-5p, let-7g-5p, miR-223-3p, miR-3615, miR-374a-5p, miR-182-5p, miR-345-5p, miR-361-3p, miR-130b-3p, miR-1299, miR-376c-3p, and miR-326. Measuring the level of miRs in the biological sample can be completed using any method. In some aspects, the level of the panel of miRs is measured using small RNA/microRNA/RNA sequencing, microarray hybridization, a northern blot, isothermal nucleic acid amplification, quantitative reverse-transcriptase PCR (qRT-PCR), or real time PCR (RT-PCR), or any combination thereof. As such, in some aspects, an RNA-hybridization probe disclosed herein can be replaced with a pair of PCR primers capable of detecting the same miR (or a cDNA created from the same miR) as the particular RNA-hybridization probe.

In some aspects, the level of the panel of miRs is measured by contacting the biological sample (or a sample comprising miRs isolated from the biological sample) with one or more RNA-hybridization probes. In some aspects, the level of the panel of miRs is measured using a microfluidic array comprising a plurality of RNA-hybridization probes (e.g., a microfluidic array disclosed herein). In some aspects, the level of a particular miR in a sample is determined by measuring the level of a detectable marker on the RNA hybridization probe.

In some aspects, the plurality of RNA-hybridization probes comprises an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326. In some aspects, the plurality of RNA-hybridization probes consists of an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326. In some aspects, the plurality of RNA-hybridization probes consists essentially of an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326.

In some aspects, the method further comprises determining an ACR signature score. In some aspects, the ACR signature score is determined according to the following formula:

$$ACR signature score = 251.89 - ( a ) * ln [ miR - 30 e - 5 p ] - ( b ) * ln [ let - 7 g - 5 p ] - ( c ) * ln [ miR - 223 - 3 p ] + ( d ) * ln [ miR - 3615 ] + ( e ) * ln [ miR - 374 a - 5 p ] + ( f ) * ln [ miR - 182 - 5 p ] - ( g ) * ln [ miR - 345 - 5 p ] + ( h ) * ln [ miR - 361 - 3 p ] - ( i ) * ln [ miR - 130 b - 3 p ] - ( j ) * ln [ miR - 1299 ] - ( k ) * ln [ miR - 376 c - 3 p ] - ( l ) * ln [ miR - 326 ] \qquad Formula I$$

$$Using reads per million data with + 10 added to each count to handle \\ non - zero values . Ln represents the natural log .$$

wherein: (a)=any number from 23 and 33; (b)=any number from 0.14 and 0.24; (c)=any number from 2.5 and 7.5; (d)=any number from 2 and 6; (e)=any number from 4 and 9; (f)=any number from 2 and 6; (g)=any number from 0.5 and 5; (h)=any number from 22 and 32; (i)=any number from 1 and 5; (j)=any number from 3 and 10; (k)=any number from 4 and 11; and (l)=any number from 6 and 14; and wherein “[X]” refers to the level of “X” in the biological sample, e.g., as represented by the number of reads per sample (per million data) by a sequencer.

In some aspects, (a)=any number from 26 to 31; (b)=any number from 0.16 to 0.21; (c)=any number from 4 to 6; (d)=any number from 3 to 5; (e)=any number from 6 to 7.5; (f)=any number from 3 to 5; (g)=any number from 1 to 3; (h)=any number from 25 to 28; (i)=any number from 2 to 4; (j)=any number from 5 to 7; (k)=any number from 7 to 9; and (l)=any number from 10 to 12.

In some aspects, (a)=about 28.90; (b)=about 0.19; (c)=about 5.46; (d)=about 4.77; (e)=about 6.41; (f)=about 4.41; (g)=about 2.20; (h)=about 27.69; (i)=about 3.05; (j)=about 6.17; (k)=about 7.71; and (l)=about 10.63.

In some aspects, the panel comprises one or more additional ACR miRs. In some aspects, the panel of miRs comprises miR-23a-3p, miR-30e-5p, let-7g-5p, miR-24-3p, miR-27a-3p, miR-223-3p, miR-197-3p, miR-3615, miR-374a-5p, miR-182-5p, miR-345-5p, miR-361-3p, miR-130b-3p, miR-1299, miR-323b-3p, miR-582-3p, miR-432-5p, miR-329-3p, miR-376c-3p, and miR-326; wherein the ACR score is calculated according to the following formula:

$$ACR Score = 326 + ( a ) * ln [ miR - 23 a - 3 p ] - ( b ) * ln [ miR - 30 e - 5 p ] + ( c ) * ln [ let - 7 g - 5 p ] - ( d ) * [ miR - 24 - 3 p ] + ( e ) * ln [ miR - 27 a - 3 p ] - ( f ) * ln [ miR - 223 - 3 p ] + ( g ) * [ miR - 197 - 3 p ] - ( h ) * ln [ miR - 3615 ] + ( i ) * ln [ miR - 374 a - 5 p ] + ( j ) * ln [ miR - 182 - 5 p ] - ( k ) * ln [ miR - 345 - 5 p ] + ( l ) * ln [ miR - 361 - 3 p ] - ( m ) * ln [ miR - 130 b - 3 p ] - ( n ) * ln [ miR - 1299 ] + ( o ) * ln [ miR - 323 b - 3 p ] + ( p ) * ln [ miR - 582 - 3 p ] + ( q ) * ln [ miR - 432 - 5 p ] + ( r ) * ln [ miR - 329 - 3 p ] - ( s ) * ln [ miR - 376 c - 3 p ] - ( t ) * ln [ miR - 326 ] . \qquad Formula II$$

$$Using reads per million data with + 10 added to each count to handle \\ non - zero values . Ln represents the natural log .$$

wherein: (a)=any number from 1 to 7; (b)=any number from 26 to 36; (c)=any number from 1 to 5; (d)=any number from 8 to 18; (e)=any number from 1 to 7; (f)=any number from 4 to 14; (g)=any number from 3 to 13; (h)=any number from 0.1 to 1; (i)=any number from 2 to 12; (j)=any number from 1 to 7; (k)=any number from 1 to 7; (l)=any number from 18 to 28; (m)=any number from 2 to 8; (n)=any number from 3 to 9; (o)=any number from 0.1 to 1; (p)=any number from 2 to 8; (q)=any number from 1 to 7; (r)=any number from 0.5 to 3.5; (s)=any number from 5 to 15; and (t)=any number from 6 to 16; and wherein “[X]” refers to the level of “X” in the biological sample, e.g., as represented by the number of reads per sample (per million data) by a sequencer.

In some aspects, (a)=any number from 2 to 5; (b)=any number from 30 to 33; (c)=any number from 1.5 to 3; (d)=any number from 11 to 15; (e)=any number from 2 to 5; (f)=any number from 7 to 11; (g)=any number from 6 to 10; (h)=any number from 0.18 to 0.38; (i)=any number from 5 to 9; (j)=any number from 2 to 5; (k)=any number from 2 to 5; (l)=any number from 20 to 25; (m)=any number from 3 to 6; (n)=any number from 4.5 to 6.5; (o)=any number from 0.5 to 1; (p)=any number from 3 to 6; (q)=any number from 2 to 5; (r)=any number from 1 to 3; (s)=any number from 8 to 12; and (t)=any number from 9 to 14.

In some aspects, (a)=about 3.64; (b)=about 31.79; (c)=about 2.10; (d)=about 13.49; (e)=about 3.75; (f)=about 9.30; (g)=about 8.18; (h)=about 0.28; (i)=about 7.51; (j)=about 3.88; (k)=about 3.59; (l)=about 23.57; (m)=about 4.58; (n)=about 5.73; (o)=about 0.71; (p)=about 4.69; (q)=about 3.32; (r)=about 1.90; (s)=about 10.12; and (t)=about 11.78.

In some aspects, a subject is identified as experiencing an ACR if the ACR score is equal to or higher than about 65.

In some aspects, a subject is identified as at risk of experiencing an ACR if the ACR score is equal to or higher than about 45. In some aspects, a subject is identified as at risk of experiencing an ACR if the ACR score is equal to or higher than about 50. In some aspects, a subject is identified as at risk of experiencing an ACR if the ACR score is from about 50 to about 60. In some aspects, a subject is identified as at risk of experiencing an ACR if the ACR score is from about 50 to about 55. In some aspects, a subject is identified as at risk of experiencing an ACR if the ACR score is equal to or higher than about 51. In some aspects, a subject is identified as at risk of experiencing an ACR if the ACR score is equal to or higher than about 52. In some aspects, a subject is identified as at risk of experiencing an ACR if the ACR score is equal to or higher than about 53. In some aspects, a subject is identified as at risk of experiencing an ACR if the ACR score is equal to or higher than about 54. In some aspects, a subject is identified as at risk of experiencing an ACR if the ACR score is equal to or higher than about 55. In some aspects, a subject is identified as at risk of experiencing an ACR if the ACR score is equal to or higher than about 56. In some aspects, a subject is identified as at risk of experiencing an ACR if the ACR score is equal to or higher than about 57. In some aspects, a subject is identified as at risk of experiencing an ACR if the ACR score is equal to or higher than about 58. In some aspects, a subject is identified as at risk of experiencing an ACR if the ACR score is equal to or higher than about 59. In some aspects, a subject is identified as at risk of experiencing an ACR if the ACR score is equal to or higher than about 60.

In some aspects, a subject having an ACR score equal to or higher than about 65 is administered an immunosuppressive therapy. In some aspects, the immunosuppressive therapy is administered to the subject after the subject is identified as having a score equal to or higher than about 65. In some aspects, the immunosuppressive therapy comprises administering a therapy selected from the group consisting of a corticosteroid, an anti-thymocyte globulin, tacrolimus, cyclosporine, sirolimus, everolimus, myocophenolate mofeitil, azathioprine, tocilizumab, belatacept, and any combination thereof. In some aspects, a subject having an ACR score equal to or higher than about 65 is subjected to an endomyocardial biopsy. In some aspects, the endomyocardial biopsy is executed after the subject is identified as having an ACR score equal to or higher than about 65. In some aspects, a subject having an ACR score less than 65 is not subjected to an endomyocardial biopsy.

### IV.A.2. AMR

In some aspects, the method comprises identifying a subject as experiencing or at risk of experiencing an AMR, wherein the method comprises measuring the level of a panel of miRs in a biological sample obtained from the subject, wherein the panel of miRs comprises miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, and miR-589-5p. Measuring the level of miRs in the biological sample can be completed using any methods. In some aspects, the level of the panel of miRs is measured using small RNA/microRNA/RNA sequencing, microarray hybridization, a northern blot, isothermal nucleic acid amplification, quantitative reverse-transcriptase PCR (qRT-PCR), or real time PCR (RT-PCR), or any combination thereof.

In some aspects, the level of the panel of miRs is measured by contacting the biological sample (or a sample comprising miRs isolated from the biological sample) with one or more RNA-hybridization probes. In some aspects, the level of the panel of miRs is measured using a microfluidic array comprising a plurality of RNA-hybridization probes (e.g., a microfluidic array disclosed herein).

In some aspects, the plurality of RNA-hybridization probes comprises an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to miR-589-5p. In some aspects, the plurality of RNA-hybridization probes consists of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to miR-589-5p. In some aspects, the plurality of RNA-hybridization probes consists essentially of an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to miR-589-5p.

In some aspects, the method further comprises determining an AMR signature score. In some aspects, the AMR signature score is determined according to the following formula:

$$AMR signature score = 222.41 - ( a ) * ln [ miR - 23 a - 3 p ] - ( b ) * ln [ miR - 484 ] - ( c ) * ln [ miR - 340 - 5 p ] + ( d ) * ln [ miR - 193 a - 5 p ] - ( e ) * ln [ miR - 215 - 5 p ] + ( f ) * ln [ miR - 142 - 3 p ] - ( g ) * ln [ miR - 374 a - 5 p ] + ( h ) * ln [ miR - 1307 ] + ( i ) * ln [ miR - 185 - 3 p ] + ( j ) * ln [ miR - 4433 b - 3 p ] + ( k ) * ln [ miR - 130 b - 3 p ] + ( l ) * ln [ miR - 331 - 5 p ] + ( m ) * ln [ miR - 140 - 5 p ] + ( n ) * ln [ miR - 223 - 5 p ] + ( o ) * ln [ miR - 582 - 3 p ] + ( p ) * ln [ miR - 122 - 3 p ] + ( q ) * ln [ miR - 589 - 5 p ] \qquad Formula III$$

$$Using reads per million data with + 10 added to each count to handle \\ non - zero values . Ln represents the natural log .$$

wherein: (a)=any number from 23 to 28; (b)=any number from 7 to 11; (c)=any number from 1 to 6; (d)=any number from 3 to 8; (e)=any number from 6 to 11; (f)=any number from 5 to 11; (g)=any number from 1 to 5; (h)=any number from 0.5 to 4; (i)=any number from 5 to 11; (j)=any number from 6 to 13; (k)=any number from 3 to 8; (l)=any number from 0.1 to 2; (m)=any number from 1 to 4; (n)=any number from 1 to 5; (o)=any number from 0.5 to 4; (p)=any number from 0.1 to 3; and (q)=any number from 0.1 to 4; and administering an immunosuppressive therapy to the human subject identified as having an AMR signature score is equal to or higher than about 65; wherein “[X]” refers to the level of “X” in the biological sample; and wherein “[X]” refers to the level of “X” in the biological sample, e.g., as represented by the number of reads per sample (per million data) by a sequencer.

In some aspects, (a)=any number from 24 to 27; (b)=any number from 8.5 to 10.5; (c)=any number from 2 to 4; (d)=any number from 4.5 to 6.5; (e)=any number from 7.5 to 9.5; (f)=any number from 7 to 9; (g)=any number from 2 to 4; (h)=any number from 1.0 to 1.75; (i)=any number from 7 to 9; (j)=any number from 9 to 11; (k)=any number from 5 to 7; (l)=any number from 1 to 2; (m)=any number from 1.5 to 2.5; (n)=any number from 1.5 to 2.5; (o)=any number from 0.7 to 1.7; (p)=any number from 0.5 to 1.5; and (q)=any number from 1.4 to 2.4.

In some aspects, (a)=about 25.44; (b)=about 9.33; (c)=about 3.39; (d)=about 5.82; (e)=about 8.24; (f)=about 8.62; (g)=about 2.75; (h)=about 1.43; (i)=about 7.95; (j)=about 9.69; (k)=about 5.47; (l)=about 0.60; (m)=about 2.05; (n)=about 2.24; (o)=about 1.40; (p)=about 0.87; and (q)=about 1.69.

In some aspects, the panel comprises one or more additional AMR miRs. In some aspects, the panel of miRs comprises miR-143-3p, let-7b-5p, miR-10b-5p, miR-23a-3p, miR-24-3p, miR-10a-5p, miR-27a-3p miR-125a-5p, miR-93-5p, let-7d-3p, miR-191-5p miR-484 miR-99b-5p, miR-340-5p, miR-1-3p, miR-193a-5p, miR-145-3p, miR-197-3p, let-7b-3p, miR-454-3p, miR-450b-5p, miR-215-5p miR-4433b-5p, miR-1249-3p, miR-142-3p, miR-145-5p, miR-374a-5p, miR-542-3p, miR-1307-3p, miR-17-5p, miR-345-5p, miR-185-3p, miR-338-5p, miR-769-5p, miR-4433b-3p, miR-130b-3p, miR-3³¹-5p, miR-140-5p, miR-223-5p, miR-582-3p, miR-122-3p, and miR-589-5p; wherein the AMR score is calculated according to the following formula:

$$AMR Score = 107.1 + ( a ) ln ( miR - 143 - 3 p ) + ( b ) ln ( let - 7 b - 5 p ) - ( c ) ln ( miR - 10 b - 5 p ) - ( d ) ln ( miR - 23 a - 3 p ) - ( e ) ln ( miR - 24 - 3 p ) + ( f ) ln ( miR - 10 a - 5 p ) + ( g ) ln ( miR - 27 a - 3 p ) + ( h ) ln ( miR - 125 a - 5 p ) - ( i ) ln ( miR - 93 - 5 p ) + ( j ) ln ( let - 7 d - 3 p ) + ( k ) ln ( miR - 191 - 5 p ) - ( l ) ln ( miR - 484 ) + ( m ) ln ( miR - 99 b - 5 p ) + ( n ) ln ( miR - 340 - 5 p ) - ( o ) ln ( miR - 1 - 3 p ) + ( p ) ln ( miR - 193 a - 5 p ) + ( q ) ln ( miR - 145 - 3 p ) - ( r ) ln ( miR - 197 - 3 p ) - ( s ) ln ( let - 7 b - 3 p ) - ( t ) ln ( miR - 454 - 3 p ) + ( u ) ln ( miR - 450 b - 5 p ) - ( v ) ln ( miR - 215 - 5 p ) - ( w ) ln ( miR - 4433 b - 5 p ) + ( x ) ln ( miR - 1249 - 3 p ) + ( y ) ln ( miR - 142 - 3 p ) - ( z ) ln ( miR - 145 - 5 p ) + ( aa ) ln ( miR - 374 a - 5 p ) - ( bb ) ln ( miR - 542 - 3 p ) + ( cc ) ln ( miR - 1307 - 3 p ) + ( dd ) ln ( miR - 17 - 5 p ) + ( ee ) ln ( miR - 345 - 5 p ) + ( ff ) ln ( miR - 185 - 3 p ) + ( gg ) ln ( miR - 338 - 5 p ) - ( hh ) ln ( miR - 769 - 5 p ) + ( ii ) ln ( miR - 4433 b - 3 p ) + ( jj ) ln ( miR - 130 b - 3 p ) - ( kk ) ln ( miR - 331 - 5 p ) + ( ll ) ln ( miR - 140 - 5 p ) - ( mm ) ln ( miR - 223 - 5 p ) + ( nn ) ln ( miR - 582 - 3 p ) + ( oo ) ln ( miR - 122 - 3 p ) - ( pp ) ln ( miR - 589 - 5 p ) . \qquad Formula IV$$

wherein: (a)=any number from 0.1 to 4; (b)=any number from 1 to 6; (c)=any number from 13 to 25; (d)=any number from 26 to 36; (e)=any number from 3 to 12; (f)=any number from 6 to 16; (g)=any number from 9 to 20; (h)=any number from 1 to 7; (i)=any number from 0.1 to 4; (j)=any number from 25 to 35; (k)=any number from 0.25 to 2.25; (l)=any number from 11 to 22; (m)=any number from 3 to 10; (n)=any number from 0.5 to 5; (o)=any number from 1 to 6; (p)=any number from 1 to 8; (q)=any number from 1 to 8; (r)=any number from 4 to 13; (s)=any number from 1 to 7; (t)=any number from 5 to 15; (u)=any number from 1 to 10; (v)=any number from 1 to 5; (w)=any number from 1 to 6; (x)=any number from 1 to 6; (y)=any number from 8 to 18; (z)=any number from 8 to 18; (aa)=any number from 1 to 6; (bb)=any number from 4 to 13; (cc)=any number from 0.25 to 2.25; (dd)=any number from 1 to 8; (ee)=any number from 1 to 7; (ff)=any number from 3 to 10; (gg)=any number from 3 to 10; (hh)=any number from 1 to 7; (ii)=any number from 5 to 15; (jj)=any number from 0.25 to 2; (kk)=any number from 1 to 7; (ll)=any number from 1 to 5; (mm)=any number from 1 to 7; (nn)=any number from 0.1 to 3; (oo)=any number from 1 to 6; and (pp)=any number from 1 to 6; and wherein “[X]” refers to the level of “X” in the biological sample, e.g., as represented by the number of reads per sample (per million data) by a sequencer.

In some aspects, (a)=any number from 1 to 2; (b)=any number from 3 to 4; (c)=any number from 19 to 22; (d)=any number from 31 to 34; (e)=any number from 6 to 9; (f)=any number from 10 to 13; (g)=any number from 11 to 16; (h)=any number from 3 to 5; (i)=any number from 1 to 2; (j)=any number from 28 to 32; (k)=any number from 0.5 to 1.5; (l)=any number from 15 to 18; (m)=any number from 5 to 7; (n)=any number from 1.5 to 2.5; (o)=any number from 2.5 to 3.5; (p)=any number from 4 to 5; (q)=any number from 4 to 5; (r)=any number from 7 to 10; (s)=any number from 3 to 5; (t)=any number from 7 to 11; (u)=any number from 4.5 to 7; (v)=any number from 2 to 4; (w)=any number from 2.5 to 4.5; (x)=any number from 2 to 5; (y)=any number from 11 to 14; (z)=any number from 11 to 14; (aa)=any number from 2.25 to 4.25; (bb)=any number from 7 to 10; (cc)=any number from 0.75 to 2; (dd)=any number from 3 to 5.5; (ee)=any number from 2 to 5; (ff)=any number from 5 to 9; (gg)=any number from 5 to 9; (hh)=any number from 2 to 5; (ii)=any number from 7 to 12; (jj)=any number from 0.25 to 1.5; (kk)=any number from 2 to 5; (ll)=any number from 2 to 4; (mm)=any number from 3 to 5; (nn)=any number from 0.5 to 1.75; (oo)=any number from 2 to 4; and (pp)=any number from 2 to 4.

In some aspects, (a)=about 1.59; (b)=about 3.4; (c)=about 20.35; (d)=about 32.4; (e)=about 7.89; (f)=about 11.93; (g)=about 14.41; (h)=about 3.93; (i)=about 1.39; (j)=about 30.11; (k)=about 1.15; (l)=about 16.25; (m)=about 6.21; (n)=about 2.03; (o)=about 3.07; (p)=about 4.92; (q)=about 4.49; (r)=about 8.65; (s)=about 4.08; (t)=about 9.15; (u)=about 5.62; (v)=about 2.96; (w)=about 3.48; (x)=about 3.21; (y)=about 12.86; (z)=about 13.0; (aa)=about 3.22; (bb)=about 8.56; (cc)=about 1.37; (dd)=about 4.39; (ee)=about 3.18; (ff)=about 6.47; (gg)=about 6.05; (hh)=about 3.74; (ii)=about 9.45; (jj)=about 0.69; (kk)=about 3.61; (ll)=about 2.84; (mm)=about 4.07; (nn)=about 1.11; (oo)=about 2.98; and (pp)=about 3.11.

In some aspects, a subject is identified as experiencing an AMR if the AMR score is equal to or higher than about 65. In some aspects, a subject is diagnosed with an AMR if the AMR score is equal to or higher than about 65.

In some aspects, a subject is identified as at risk of experiencing an AMR if the AMR score is equal to or higher than about 45. In some aspects, a subject is identified as at risk of experiencing an AMR if the AMR score is equal to or higher than about 50. In some aspects, a subject is identified as at risk of experiencing an AMR if the AMR score is from about 50 to about 60. In some aspects, a subject is identified as at risk of experiencing an AMR if the AMR score is from about 55 to about 60. In some aspects, a subject is identified as at risk of experiencing an AMR if the AMR score is equal to or higher than about 51. In some aspects, a subject is identified as at risk of experiencing an AMR if the AMR score is equal to or higher than about 52. In some aspects, a subject is identified as at risk of experiencing an AMR if the AMR score is equal to or higher than about 53. In some aspects, a subject is identified as at risk of experiencing an AMR if the AMR score is equal to or higher than about 54. In some aspects, a subject is identified as at risk of experiencing an AMR if the AMR score is equal to or higher than about 55. In some aspects, a subject is identified as at risk of experiencing an AMR if the AMR score is equal to or higher than about 56. In some aspects, a subject is identified as at risk of experiencing an AMR if the AMR score is equal to or higher than about 57. In some aspects, a subject is identified as at risk of experiencing an AMR if the AMR score is equal to or higher than about 58. In some aspects, a subject is identified as at risk of experiencing an AMR if the AMR score is equal to or higher than about 59. In some aspects, a subject is identified as at risk of experiencing an AMR if the AMR score is equal to or higher than about 60.

In some aspects, a subject having an AMR score equal to or higher than about 65 is administered an immunosuppressive therapy. In some aspects, the immunosuppressive therapy comprises administering a therapy selected from the group consisting of intravenous immunoglobulin, plasmapheresis, bortezomib, carfilzomib, rituximab, eculizumab, a corticosteroid, an anti-thymocyte globulin, tacrolimus, cyclosporine, sirolimus, everolimus, myocophenolate mofeitil, azathioprine, tocilizumab, belatacept, and any combination thereof. In some aspects, a subject having an AMR score equal to or higher than about 65 is subjected to an endomyocardial biopsy. In some aspects, a subject having an AMR score less than 65 is not subjected to an endomyocardial biopsy.

### IV.B. Methods of Treatment

Some aspects of the present disclosure are directed to a method of treating acute heart allograft rejection in a subject in need thereof comprising (i) identifying a subject experiencing or at risk of experiencing an acute heart allograft rejection selected from an ACR and an AMR, and (ii) administering an immunosuppressive therapy to the subject. In some aspects, the acute heart allograft rejection comprises an ACR, and the subject is administered an immunosuppressive therapy comprising administering a therapy selected from the group consisting of a corticosteroid, an anti-thymocyte globulin, tacrolimus, cyclosporine, sirolimus, everolimus, myocophenolate mofeitil, azathioprine, tocilizumab, belatacept, and any combination thereof. In some aspects, the subject is identified as experiencing or at risk of experiencing an ACR, and the subject is administered a corticosteroid. In some aspects, the subject is identified as experiencing or at risk of experiencing an ACR, and the subject is administered an anti-thymocyte globulin. In some aspects, the subject is identified as experiencing or at risk of experiencing an ACR, and the subject is administered tacrolimus. In some aspects, the subject is identified as experiencing or at risk of experiencing an ACR, and the subject is administered cyclosporine. In some aspects, the subject is identified as experiencing or at risk of experiencing an ACR, and the subject is administered sirolimus. In some aspects, the subject is identified as experiencing or at risk of experiencing an ACR, and the subject is administered everolimus. In some aspects, the subject is identified as experiencing or at risk of experiencing an ACR, and the subject is administered myocophenolate mofeitil. In some aspects, the subject is identified as experiencing or at risk of experiencing an ACR, and the subject is administered azathioprine. In some aspects, the subject is identified as experiencing or at risk of experiencing an ACR, and the subject is administered tocilizumab. In some aspects, the subject is identified as experiencing or at risk of experiencing an ACR, and the subject is administered belatacept. In some aspects, the subject is identified as experiencing or at risk of experiencing an ACR, and the subject is treated with a repeat heart transplant surgery.

In some aspects, the acute heart allograft rejection comprises AMR, and the subject is administered an immunosuppressive therapy comprising administering a therapy selected from the group consisting of intravenous immunoglobulin, plasmapheresis, bortezomib, carfilzomib, rituximab, eculizumab, a corticosteroid, an anti-thymocyte globulin, tacrolimus, cyclosporine, sirolimus, everolimus, myocophenolate mofeitil, azathioprine, tocilizumab, belatacept, and any combination thereof. In some aspects, the subject is identified as experiencing or at risk of experiencing an AMR, and the subject is administered intravenous immunoglobulin. In some aspects, the subject is identified as experiencing or at risk of experiencing an AMR, and the subject is administered plasmapheresis. In some aspects, the subject is identified as experiencing or at risk of experiencing an AMR, and the subject is administered bortezomib. In some aspects, the subject is identified as experiencing or at risk of experiencing an AMR, and the subject is administered carfilzomib. In some aspects, the subject is identified as experiencing or at risk of experiencing an AMR, and the subject is administered rituximab. In some aspects, the subject is identified as experiencing or at risk of experiencing an AMR, and the subject is administered eculizumab. In some aspects, the subject is identified as experiencing or at risk of experiencing an AMR, and the subject is administered a corticosteroid. In some aspects, the subject is identified as experiencing or at risk of experiencing an AMR, and the subject is administered an anti-thymocyte globulin. In some aspects, the subject is identified as experiencing or at risk of experiencing an AMR, and the subject is administered tacrolimus. In some aspects, the subject is identified as experiencing or at risk of experiencing an AMR, and the subject is administered cyclosporine. In some aspects, the subject is identified as experiencing or at risk of experiencing an AMR, and the subject is administered sirolimus. In some aspects, the subject is identified as experiencing or at risk of experiencing an AMR, and the subject is administered everolimus. In some aspects, the subject is identified as experiencing or at risk of experiencing an AMR, and the subject is administered myocophenolate mofeitil. In some aspects, the subject is identified as experiencing or at risk of experiencing an AMR, and the subject is administered azathioprine. In some aspects, the subject is identified as experiencing or at risk of experiencing an AMR, and the subject is administered tocilizumab. In some aspects, the subject is identified as experiencing or at risk of experiencing an AMR, and the subject is administered belatacept. In some aspects, the subject is identified as experiencing or at risk of experiencing an AMR, and the subject is treated with a repeat heart transplant surgery.

### IV.C. System for Detecting an Allograft Rejection

Some aspects of the present disclosure provide methods of identifying a subject experiencing or at risk of developing acute allograft heart rejection. However, the techniques used to identify the various miRs used in these methods can be applied to identifying miRs that may be indicative of other types of organ rejection. As such, some aspects of the present disclosure are directed to determining the miR transcriptome of heart transplant recipients, distinguishing miR expression in the setting of ACR and AMR, and developing distinct miR panels that could be used to non-invasively diagnose ACR and AMR. Furthermore, aspects described herein allow for using genomic biomarker testing to permit patients to begin specific treatment pathways based on the subtype of rejection. Moreover, aspects described herein create individual ACR and AMR miR gene expression scores that allow for clinical interpretation and non-invasive diagnosis of acute rejection. Since the scores are unique to ACR or AMR, if elevated, they can lead to targeted therapy while awaiting results from other diagnostic testing.

In this regard, aspects described herein provide for diagnosing a heart transplant rejection using merely a blood test (“liquid biopsy”).

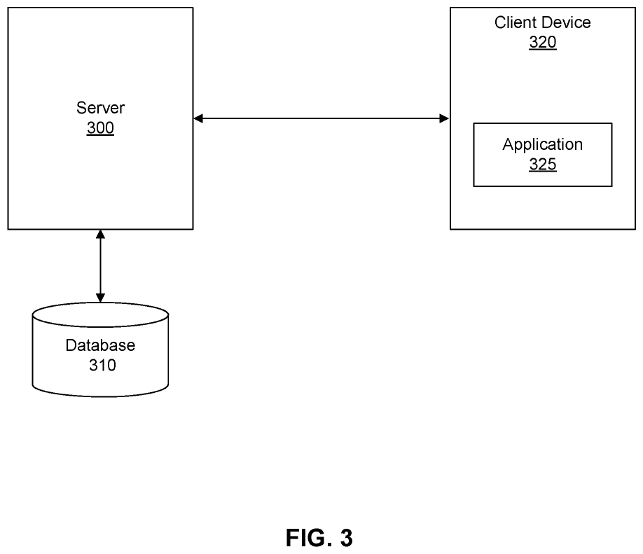

FIG. 3 is a block diagram of a system for detecting an allograft rejection based on miRs, according to some aspects. The system may include a server 300, database 310, and client device 320. The devices of the system may be connected through a network. For example, the devices of the system may be connected through wired connections, wireless connections, or a combination of wired and wireless connections. In an example aspect, one or more portions of the network may be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

In some aspects, server 300 and database 310 may reside in a cloud-computing environment. In other aspects, server 300 may reside in a cloud-computing environment, while database 310 resides outside the cloud-computing environment. Furthermore, in other aspects, server 300 may reside outside the cloud-computing environment, while database 310 resides in the cloud-computing environment.

Server 300 may be configured to execute one or more applications to identify a likelihood of allograft rejection based on miRs. Database 310 may be configured to store structured and unstructured data. Server 300 may store and retrieve data from database 310 to identify the likelihood of allograft rejection based on miRs.

Client device 320 may be in communication with server 300. Client device 320 may be configured to execute application 325 to communicate with server 300. Application 325 may be used to transmit requests to server 300 to identify a likelihood of allograft rejection based on miRs for one or more patients. Application 325 may include a user interface. The user interface may be used to transmit the requests to server 300. Furthermore, the user interface may render a response received from the server 300. Client device 320 may be operated by users including but not limited to patients, healthcare professionals, insurance companies, etc.

In an aspect, client device 320 and server 300 are combined into the same device, such that operations described herein as performed by the server execute directly on the client device, and the client device itself is in communication with database 310.

### IV.C.1. miR Panel Generation

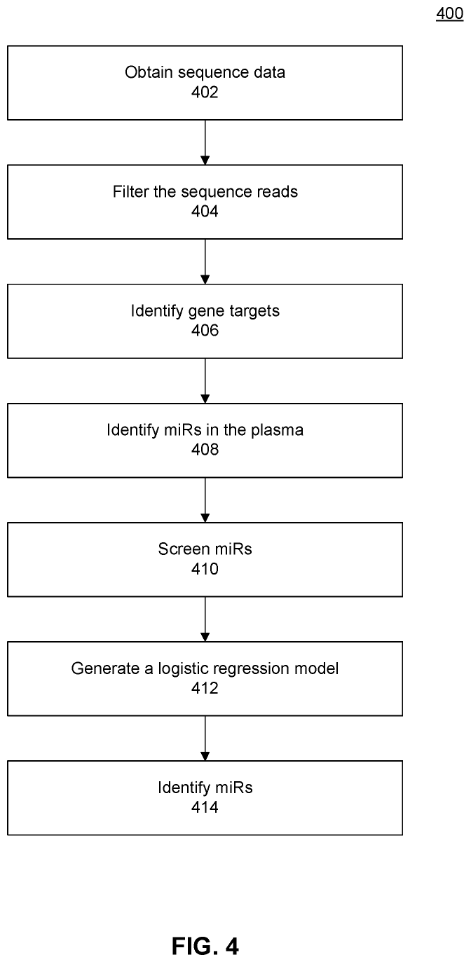

FIG. 4 is a flowchart illustrating the process of developing a miR panel, according to some aspects. Though applied to miR panels associated with acute heart allograft rejection herein, the methods described herein can be applied broadly to develop miR panels for other organ rejections. Method 400 may be performed by processing logic that may comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously or in a different order than shown in FIG. 4, as will be understood by a person of ordinary skill in the art

Method 400 shall be described with reference to FIG. 3. However, method 400 is not limited to that example aspect.

In 402, sequence data for a plurality of subjects is obtained. In some aspects, server 300 may sequence the RNA from samples obtained from subjects to generate sequence reads. The subjects may include patients and/or control subjects. In some aspects related to developing a miR panel for identifying organ transplant rejection, the samples may be blood, urine, cerebrospinal fluid, seminal fluid, saliva, sputum, stool, and tissue. The sequence data is obtained from any suitable internal or external data sources (e.g., database 310). As a non-limiting example, sequence data may be obtained from data sources associated with Genomic Research Alliance for Transplantation (GRAfT), Cedars-Sinai Medical Center, University of Utah, Stanford University Heart Transplant Biobank, Outcomes AlloMap Registry (OAR), Donor-Derived Cell-Free DNA-Outcomes Allomap Registry (D-OAR), Surveillance HeartCare Outcomes Registry (SHORE), Donor-Derived Cell-free DNA to DETect REjection in Cardiac Transplantation (DETECT), PROTECT Registry, INTERHEART Study, DNA-Based Transplant Rejection Test (DTRT) study, Clinical Trials in Organ Transplantation, etc. Furthermore, blood samples may be collected from a subject or patient under surveillance or clinically indicated testing. The miRs may be blood-based. For patients that are undergoing EMB, the blood samples may be collected before the procedure. The venous blood may be collected into containers, e.g., Streck Cell-Free DNA tubes, and processed to isolate the plasma. The plasma may be stored at an appropriate temperature, such as −80° C. The RNA may be extracted from the plasma using known extraction techniques, such as the MagMAX mirVana isolation kit (Thermo Fisher Scientific, Gaithersburg MD). After elution, library preparation may be performed using a library preparation tool, such as the NEXTFLEX Small RNA-Seq Kit v3 (PerkinElmer Inc, Shelton CT). The prepared RNA may then be sequenced using a sequencing machine, such as the NEXTSEQ 500 (Illumina Inc., San Diego CA) using, for example, 50 base pair (bp) single-end reads. In an aspect, server 300 may implement the sequencing machine to sequence the RNA. The sequencing may generate, for example, ˜11 million sequence reads per sample.

In some aspects, other sequencing methodologies may be used. For example, server 300 may sequence the RNA in the sample using Complementary DNA sequencing, Small RNA/non-coding RNA sequencing, Direct RNA sequencing, Single-molecule real-time RNA sequencing, Single-cell RNA sequencing (scRNA-Seq), etc. In some aspects, the sequences may be obtained from a previously populated database such that server 300 does not perform the actual sequencing but rather obtains sequence data from the database.

In some aspects, the sequence reads may be identified based on annotating the sequence reads using PCR, arrays, probe-based assays, iNAAT, CRISPR, or other molecular testing methods.

In 404, server 300 filters the sequence reads by removing one or more sequence reads from the generated sequence reads. For example, sequence FASTQ files corresponding to the sequence reads may be processed to remove 3′ adapter barcode sequences, random-barcode sequences, UniVec contaminants, and reads <15 base pairs (bp). Furthermore, high-quality filtered sequences from the sequence reads may be endogenously aligned using miRbase v22, GENCODE v24 for the reference human genome 38, and gtRNAdb v16. Samples with insufficient miR reads (<1,000,000 reads per sample) of good quality may be excluded from the analysis. Small RNA sequence data in the plurality of sequence reads may be analyzed using the Extracellular RNA Communication Consortium (ERCC) RNA pipeline (extracellular RNA processing tool, exceRpt).

To eliminate or otherwise mitigate batch effect across small RNA sequencing runs, server 300 may perform a principal component analysis (PCA) on the sequence reads (excluding the one or more removed sequence reads). This PCA may use, for example, Caenorhabditis elegans sample spike-ins and a pooled plasma control from unrelated human donors. A batch effect occurs when non-biological factors in an experiment cause changes in the data produced by the experiment. Server 300 may exclude outlier samples from further analysis.

In 406, server 300 identifies gene targets in the filtered sequence reads of miRs. The gene targets are identified as implicated in ACR or AMR. For example, server 300 may identify gene targets of miRs implicated in ACR or AMR using miRTarBase v8.0. Server 300 may include mRNA targets validated through, e.g., reporter assays, western blot, or microarray experiments with overexpression or knockdown of miRs in the analysis. Server 300 may execute the network analysis and data visualization using an interactive web tool such as, for example, MIENTURNET. Server 300 may distinguish biological pathways potentially regulated by the differentially expressed miRs by searching a publicly available database, such as the Reactome database. In some aspects, the publicly available database may be part of database 310.

In 408, server 300 identifies miRs in the sample based on the identified gene targets. As a non-limiting example, server 300 may identify ˜1,900 expressed miRs in plasma. Server 300 may filter out lowly expressed miRs (e.g., miRs with less than 100 average mapped reads across all samples). In addition, server 300 removes miR 486-5p and miR-451a, which are red blood cell-derived miRs. Server 300 includes the remaining miRs (e.g., ˜350 miRs) in the analysis.

In 410, server 300 screens the miRs (e.g., the remaining ˜350 miRs) to identify differentially expressed miRs while adjusting for clinical covariates (e.g., age, sex, race, body-mass index) using a differential gene expression analysis tool, such as DESeq2. According to aspects, the differentially expressed miRs may be screened based on their correspondence to ACR or AMR. By doing so, server 300 identifies the differentially expressed miRs associated with a specific outcome that may be identified from the miR transcriptome. Server 300 may adjust the blood group in a subset of patients with minimal change in miR profile. Alternatively, server 300 may adjust the data for age, sex, race, and body-mass index to maximize sample size.

Approximately 50% of all protein-coding genes are under the control of miRs, and a single mRNA may be regulated by multiple miRs. Most commonly, miRs negatively regulate downstream gene expression. Gene expression signatures in ACR and AMR may arise from the immune system response to allograft injury or directly from apoptotic cardiomyocytes and endothelial cells in the injured cardiac allograft. In this regard, differentially expressed miRs may be dysregulated in certain key pathways implicated in ACR or AMR: mTOR signaling, T-cell differentiation, interleukin signaling, DNA damage recognition, transcription regulation, TGF-β signaling, tumor necrosis factor (TNF) signaling, toll-like receptor cascades, T-cell receptor signaling, lymphocyte proliferation, and cell death/apoptosis.

According to an aspect for heart allograft detection, after filtering lowly expressed miRs, server 300 determined that ˜350 miRs are consistently expressed across all heart transplant patients (12 differentially expressed miRs in ACR and 27 in AMR). Using a rigorous statistical approach while controlling for clinical co-variates, 12 miR were selected for ACR and 17 for AMR. Further, these miRs had minimal correlation with each other (data not presented), suggesting that they are individually informative of rejection.

In 412, server 300 generates a first logistic regression model corresponding to the specific outcome (e.g., AMR or ACR) using the identified miRs with an unadjusted p-value of, for example, <0.10. In some aspects, server 300 fits the first logistic regression model with a LASSO penalty using the identified miRs. Within the LASSO analysis, server 300 may log-transform normalized miR counts to approximate normality. The log counts for each miR may be standardized, such as having a mean of zero and a variance of one. The tuning parameter of the LASSO penalty may be selected by server 300 to minimize model deviance based on, for example, 10-fold cross-validation.

In 414, server 300 identifies one or more miRs from the identified miRs using the logistic-LASSO regression. The one or more miRs constitute the miR panel used to diagnose AMR or ACR. For example, server 300 generates a miR panel for diagnosing AMR based on differentially expressed miRs corresponding to AMR. In another example, server 300 generates a different miR panel for diagnosing ACR based on differentially expressed miRs corresponding to ACR. Server 300 may generate receiver operating characteristic (ROC) curves and calculate the area under the curves (AUC) to assess the performance of the identified set of miRs. The model may also be independently validated for ACR and AMR.

In light of the above, using the identified one or more miRs (corresponding to ACR or AMR) to diagnose AMR or ACR increases the computational efficiency and reduces the amount of time required to diagnose a transplant rejection. Specifically, rather than having to analyze all of the miRs in a given sample, specific miRs may be targeted in a sample. This significantly reduces the amount of data to be processed. As a result, this increases computational efficiency in diagnosing a possible transplant rejection.

Moreover, being able to target specific miRs in a sample provides increased accuracy in diagnosing a transplant rejection using less data. For example, attempting to diagnose the possible transplant rejection from a given sample without the specific miRs may provide false indications of possible transplant rejection or may altogether miss a possible transplant rejection. As such, by using the identified one or more miRs for diagnosing AMR or ACR allows for greater accuracy in diagnosing the transplant rejection.

### IV.C.2. Generation of the Model and Scores

Server 300 generates a second logistic regression model for ACR and AMR using the read counts per million data (plus 10 for each read count for non-zero values) for the one or more miRs selected by the logistic-LASSO regression (e.g., the miR panel), as applied to the subjects identified for the first logistic regression model. The read counts per million data may be based on the miR panel identified for each subject. The ordinary logistic regression estimates are prone to bias due to the low sample size and the large observed difference in expression of some miRs in patients and controls (which causes the likelihood function to be flat near the maximum), particularly for AMR. In this scenario, server 300 may use a bias-reduced maximum likelihood estimator for the final logistic regression model parameters.

ROC curves and AUC statistics may be generated using, e.g., 10-fold cross-validation to assess the predictive ability of the miRs. A Youden index may be used to identify a threshold to maximize test performance. Test performance characteristics in the validation may include sensitivity, specificity, negative predictive value (NPV), and positive predictive value (PPV).

Server 300 may identify distinct ACR and AMR rejection score thresholds based on population divisions shown by the second logistic regression model. Server 300 may identify the score thresholds based on the counts per million data for the miRs panel for each subject, as plotted on the second logistic regression model. These distinct ACR and AMR rejection score thresholds are based on the miR expression data for each patient sample. These score thresholds can be used to facilitate the interpretation of blood miR expression data to support clinical decision-making about the likelihood of ACR or AMR.

### IV.C.3. Development of Algorithm for Calculating ACR or AMR in a Patient

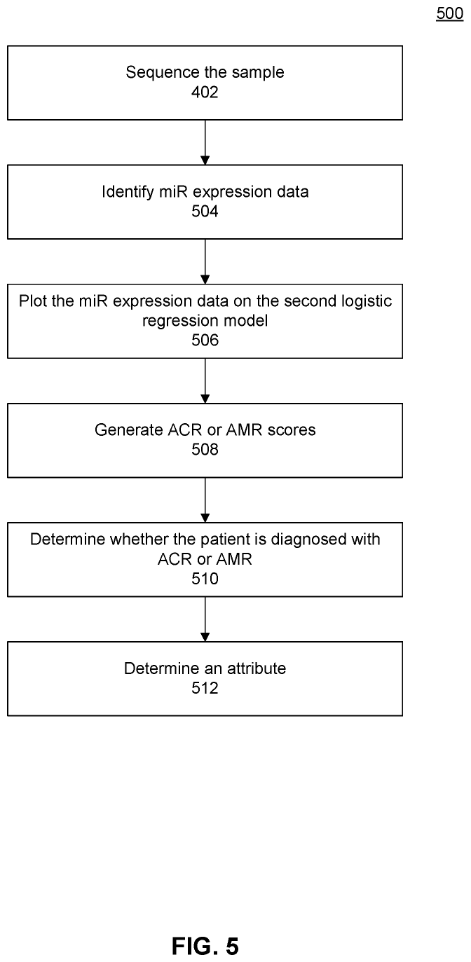

FIG. 5 is a flowchart illustrating a process for identifying a heart allograft rejection in a patient, according to some aspects. Method 500 may be performed by processing logic that may comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously or in a different order than shown in FIG. 5, as will be understood by a person of ordinary skill in the art

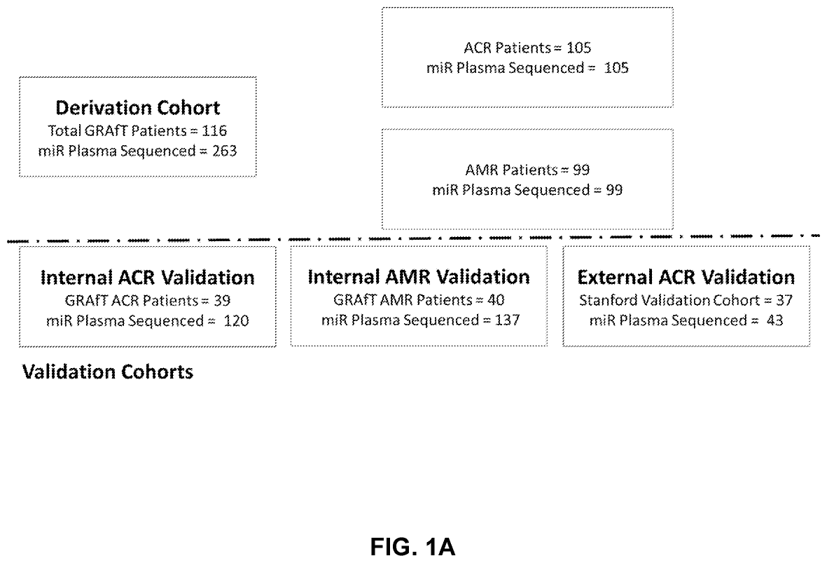

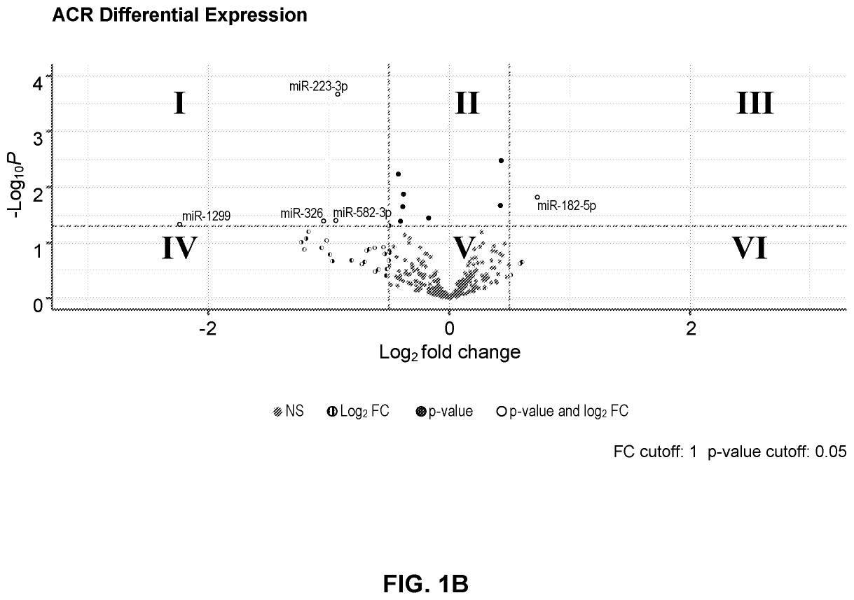

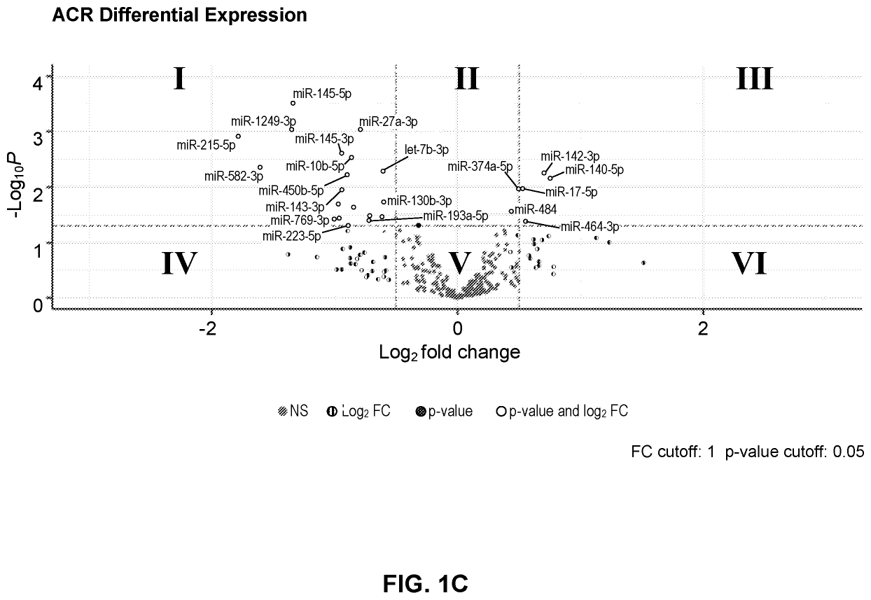

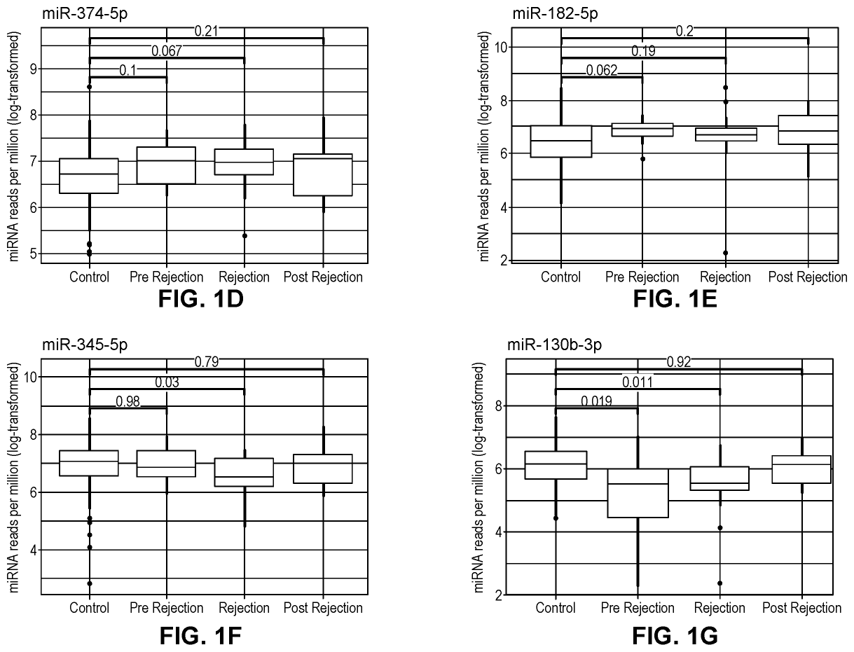

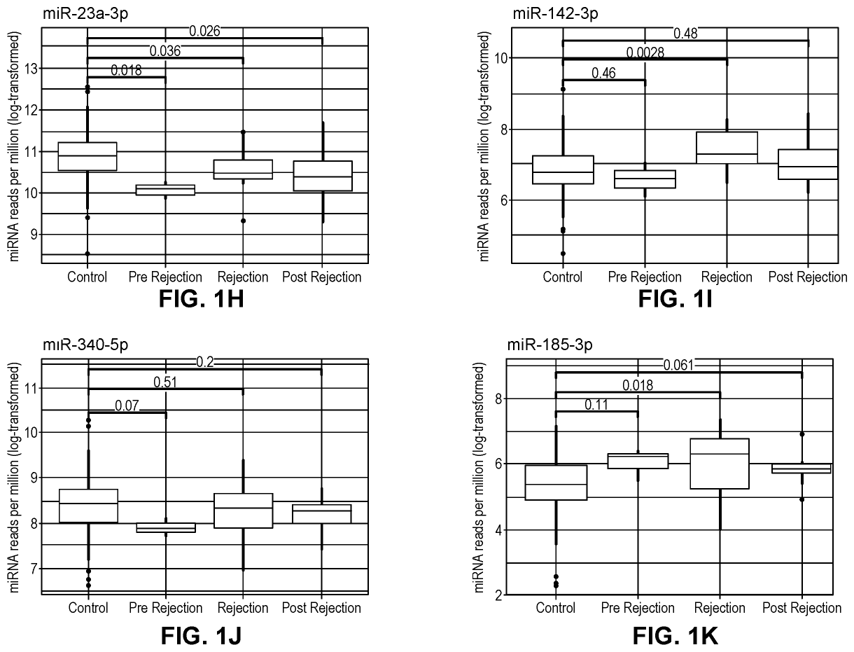

Method 500 shall be described with reference to FIG. 1. However, method 500 is not limited to that example aspect.

In 502, server 300 sequences a sample obtained from a patient to generate sequence reads. The patient is a heart transplant recipient. The sample is sequenced as described above with respect to method 400 (402).

In 504, server 300 identifies patient-specific miR expression data from the sequence reads based on the miR panel, as identified in method 400. The miR panel may be for diagnosing ACR or AMR. In this regard, server 300 identifies, from the patient's sequence reads, the patient's counts per million data for each mIR in the miR panel.

In 506, server 300 plots the patient-specific miR expression data against the second logistic regression model (i.e., the logistic regression model based on the read counts per million data for the one or more miRs in the selected mIR panel).

In 508, server 300 generates an ACR score or an AMR score specific to the patient. Specifically, if server 300 uses the miR panel to diagnose ACR in 504, server 300 generates an ACR score. Alternatively, if server 300 uses the miR panel to diagnose AMR in 504, server 300 generates an AMR score. The scores may be generated based on the miR expression data for each patient sample. This score can facilitate the interpretation of blood miR expression data to support clinical decision-making about the likelihood of ACR or AMR and facilitate the selection of treatment pathways that should be considered based on the subtype of rejection. By generating the scores, server 300 may non-invasively diagnose ACR or AMR using the miR panel and the logistic regression model.

To calculate the distinct ACR or AMR rejection scores, a coefficient is identified based on the patient-specific miR expression data plotted against the second logistic regression model. The coefficient is multiplied to each natural log-transformed miR. A nominal value, such as a value of 10, is added to the sequence reads per million data to reduce computational errors or biases in case there are any zero values. This creates a weighted score based on the relative importance of each microRNA in predicting ACR or AMR. The ACR or AMR scores may be scaled from 0-100. An individual miR ACR and AMR score may be calculated for each blood-based sample.

A threshold may be identified such that patients having scores at or above the threshold may be considered as having or risk of developing ACR or AMR rejection, while patients with scores below the threshold may be considered as unlikely to have or be at risk of developing ACR or AMR rejection. To identify a specific score threshold, ROC curves may be generated, and a Youden's Index may be used to identify the threshold of the score to maximize the AUC and test performance characteristics. In an example implementation according to an aspect of the disclosure, the threshold score for AMR and ACR that maximized sensitivity and specificity was calculated to be 65.

In an example implementation, the ACR score threshold of 65 led to an AUC of 0.85 (95% CI: 0.78-0.92), the associated test characteristics were a sensitivity of 86%, specificity of 76%, NPV of 98%, and PPV of 30%. For AMR the AUC was 0.83 (95% CI: 0.77-0.89). An AMR score threshold of 65 led to a sensitivity of 89%, specificity of 63%, NPV of 97%, and PPV of 29%. The score threshold can be increased or decreased in some aspects to maximize test sensitivity and specificity.

As a non-limiting example, the ACR score may be calculated as follows:

$$ACR SCORE = 251.89 - ( a ) * ln [ miR - 30 e - 5 p ] - ( b ) * ln [ let - 7 g - 5 p ] - ( c ) * ln [ miR - 223 - 3 p ] + ( d ) * ln [ miR - 3615 ] + ( e ) * ln [ miR - 374 a - 5 p ] + ( f ) * ln [ miR - 182 - 5 p ] - ( g ) * ln [ miR - 345 - 5 p ] + ( h ) * ln [ miR - 361 - 3 p ] - ( i ) * ln [ miR - 130 b - 3 p ] - ( j ) * ln [ miR - 1299 ] - ( k ) * ln [ miR - 376 c - 3 p ] - ( l ) * ln [ miR - 326 ]$$

wherein: (a)=any number from 23 and 33; (b)=any number from 0.14 and 0.24; (c)=any number from 2.5 and 7.5; (d)=any number from 2 and 6; (e)=any number from 4 and 9; (f)=any number from 2 and 6; (g)=any number from 0.5 and 5; (h)=any number from 22 and 32; (i)=any number from 1 and 5; 0)=any number from 3 and 10; (k)=any number from 4 and 11; and (l)=any number from 6 and 14; and wherein “[X]” refers to the level of “X” in the biological sample, e.g., as represented by the number of reads per sample (per million data) by a sequencer.

In some aspects, (a)=any number from 26 to 31; (b)=any number from 0.16 to 0.21; (c)=any number from 4 to 6; (d)=any number from 3 to 5; (e)=any number from 6 to 7.5; (f)=any number from 3 to 5; (g)=any number from 1 to 3; (h)=any number from 25 to 28; (i)=any number from 2 to 4; (j)=any number from 5 to 7; (k)=any number from 7 to 9; and (l)=any number from 10 to 12.

In some aspects, (a)=about 28.90; (b)=about 0.19; (c)=about 5.46; (d)=about 4.77; (e)=about 6.41; (f)=about 4.41; (g)=about 2.20; (h)=about 27.69; (i)=about 3.05; (j)=about 6.17; (k)=about 7.71; and (l)=about 10.63.

As another non-limiting example, the ACR score may be calculated as follows:

$$ACR Score = 326 + ( a ) * ln [ miR - 23 a - 3 p ] - ( b ) * ln [ miR - 30 e - 5 p ] + ( c ) * ln [ let - 7 g - 5 p ] - ( d ) * [ miR - 24 - 3 p ] + ( e ) * ln [ miR - 27 a - 3 p ] - ( f ) * ln [ miR - 223 - 3 p ] + ( g ) * ln [ miR - 197 - 3 p ] - ( h ) * ln [ miR - 3615 ] + ( i ) * ln [ miR - 374 a - 5 p ] + ( j ) * ln [ miR - 182 - 5 p ] - ( k ) * ln [ miR - 345 - 5 p ] + ( l ) * ln [ miR - 361 - 3 p ] - ( m ) * ln [ miR - 130 b - 3 p ] - ( n ) * ln [ miR - 1299 ] + ( o ) * ln [ miR - 323 b - 3 p ] + ( p ) * ln [ miR - 582 - 3 p ] + ( q ) * ln [ miR - 432 - 5 p ] + ( r ) * ln [ miR - 329 - 3 p ] - ( s ) * ln [ miR - 376 c - 3 p ] - ( t ) * ln [ miR - 326 ]$$

wherein: (a)=any number from 1 to 7; (b)=any number from 26 to 36; (c)=any number from 1 to 5; (d)=any number from 8 to 18; (e)=any number from 1 to 7; (f)=any number from 4 to 14; (g)=any number from 3 to 13; (h)=any number from 0.1 to 1; (i)=any number from 2 to 12; 0)=any number from 1 to 7; (k)=any number from 1 to 7; (l)=any number from 18 to 28; (m)=any number from 2 to 8; (n)=any number from 3 to 9; (o)=any number from 0.1 to 1; (p)=any number from 2 to 8; (q)=any number from 1 to 7; (r)=any number from 0.5 to 3.5; (s)=any number from 5 to 15; and (t)=any number from 6 to 16; and wherein “[X]” refers to the level of “X” in the biological sample, e.g., as represented by the number of reads per sample (per million data) by a sequencer.

In some aspects, (a)=any number from 2 to 5; (b)=any number from 30 to 33; (c)=any number from 1.5 to 3; (d)=any number from 11 to 15; (e)=any number from 2 to 5; (f)=any number from 7 to 11; (g)=any number from 6 to 10; (h)=any number from 0.18 to 0.38; (i)=any number from 5 to 9; (j)=any number from 2 to 5; (k)=any number from 2 to 5; (l)=any number from 20 to 25; (m)=any number from 3 to 6; (n)=any number from 4.5 to 6.5; (o)=any number from 0.5 to 1; (p)=any number from 3 to 6; (q)=any number from 2 to 5; (r)=any number from 1 to 3; (s)=any number from 8 to 12; and (t)=any number from 9 to 14.

In some aspects, (a)=about 3.64; (b)=about 31.79; (c)=about 2.10; (d)=about 13.49; (e)=about 3.75; (f)=about 9.30; (g)=about 8.18; (h)=about 0.28; (i)=about 7.51; (j)=about 3.88; (k)=about 3.59; (l)=about 23.57; (m)=about 4.58; (n)=about 5.73; (o)=about 0.71; (p)=about 4.69; (q)=about 3.32; (r)=about 1.90; (s)=about 10.12; and (t)=about 11.78.

As another non-limiting example, the AMR score may be calculated as follows:

$$AMR SCORE = 222.41 - ( a ) * ln [ miR - 23 a - 3 p ] - ( b ) * ln [ miR - 484 ] - ( c ) * ln [ miR - 340 - 5 p ] + ( d ) * ln [ miR - 193 a - 5 p ] - ( e ) * ln [ miR - 215 - 5 p ] + ( f ) * ln [ miR - 142 - 3 p ] - ( g ) * ln [ miR - 374 a - 5 p ] + ( h ) * ln [ miR - 1307 ] + ( i ) * ln [ miR - 185 - 3 p ] + ( j ) * ln * [ miR - 4433 b - 3 p ] + ( k ) * ln [ miR - 130 b - 3 p ] + ( l ) * ln [ miR - 331 - 5 p ] + ( m ) * ln [ miR - 140 - 5 p ] + ( n ) * ln [ miR - 223 - 5 p ] + ( o ) * ln [ miR - 582 - 3 p ] + ( p ) * ln [ miR - 122 - 3 p ] + ( q ) * ln [ miR - 569 - 5 p ]$$

wherein: (a)=any number from 23 to 28; (b)=any number from 7 to 11; (c)=any number from 1 to 6; (d)=any number from 3 to 8; (e)=any number from 6 to 11; (f)=any number from 5 to 11; (g)=any number from 1 to 5; (h)=any number from 0.5 to 4; (i)=any number from 5 to 11; (j)=any number from 6 to 13; (k)=any number from 3 to 8; (l)=any number from 0.1 to 2; (m)=any number from 1 to 4; (n)=any number from 1 to 5; (o)=any number from 0.5 to 4; (p)=any number from 0.1 to 3; and (q)=any number from 0.1 to 4; and administering an immunosuppressive therapy to the human subject identified as having an AMR signature score is equal to or higher than about 65; wherein “[X]” refers to the level of “X” in the biological sample; and wherein “[X]” refers to the level of “X” in the biological sample, e.g., as represented by the number of reads per sample (per million data) by a sequencer.

In some aspects, (a)=any number from 24 to 27; (b)=any number from 8.5 to 10.5; (c)=any number from 2 to 4; (d)=any number from 4.5 to 6.5; (e)=any number from 7.5 to 9.5; (f)=any number from 7 to 9; (g)=any number from 2 to 4; (h)=any number from 1.0 to 1.75; (i)=any number from 7 to 9; (j)=any number from 9 to 11; (k)=any number from 5 to 7; (l)=any number from 1 to 2; (m)=any number from 1.5 to 2.5; (n)=any number from 1.5 to 2.5; (o)=any number from 0.7 to 1.7; (p)=any number from 0.5 to 1.5; and (q)=any number from 1.4 to 2.4.

In some aspects, (a)=about 25.44; (b)=about 9.33; (c)=about 3.39; (d)=about 5.82; (e)=about 8.24; (f)=about 8.62; (g)=about 2.75; (h)=about 1.43; (i)=about 7.95; (j)=about 9.69; (k)=about 5.47; (l)=about 0.60; (m)=about 2.05; (n)=about 2.24; (o)=about 1.40; (p)=about 0.87; and (q)=about 1.69.

As another non-limiting example, the AMR score may be calculated as follows:

$$AMR Score = 107.1 + ( a ) ln ( miR - 143 - 3 p ) + ( b ) ln ( let - 7 b - 5 p ) - ( c ) ln ( miR - 10 b - 5 p ) - ( d ) ln ( miR - 23 a - 3 p ) - ( e ) ln ( miR - 24 - 3 p ) + ( f ) ln ( miR - 10 a - 5 p ) + ( g ) ln ( miR - 27 a - 3 p ) + ( h ) ln ( miR - 125 a - 5 p ) - ( i ) ln ( miR - 93 - 5 p ) + ( j ) ln ( let - 7 d - 3 p ) + ( k ) ln ( miR - 191 - 5 p ) - ( l ) ln ( miR - 484 ) + ( m ) ln ( miR - 99 b - 5 p ) + ( n ) ln ( miR - 340 - 5 p ) - ( o ) ln ( miR - 1 - 3 p ) + ( p ) ln ( miR - 193 a - 5 p ) + ( q ) ln ( miR - 145 - 3 p ) - ( r ) ln ( miR - 197 - 3 p ) - ( s ) ln ( let - 7 b - 3 p ) - ( t ) ln ( miR - 454 - 3 p ) + ( u ) ln ( miR - 450 b - 5 p ) - ( v ) ln ( miR - 215 - 5 p ) - ( w ) ln ( miR - 4433 b - 5 p ) + ( x ) ln ( miR - 1249 - 3 p ) + ( y ) ln ( miR - 142 - 3 p ) - ( z ) ln ( miR - 145 - 5 p ) + ( aa ) ln ( miR - 374 a - 5 p ) - ( bb ) ln ( miR - 542 - 3 p ) + ( cc ) ln ( miR - 1307 - 3 p ) + ( dd ) ln ( miR - 17 - 5 p ) + ( ee ) ln ( miR - 345 - 5 p ) + ( ff ) ln ( miR - 185 - 3 p ) + ( gg ) ln ( miR - 338 - 5 p ) - ( hh ) ln ( miR - 769 - 5 p ) + ( ii ) ln ( miR - 4433 b - 3 p ) + ( jj ) ln ( miR - 130 b - 3 p ) - ( kk ) ln ( miR - 331 - 5 p ) + ( ll ) ln ( miR - 140 - 5 p ) - ( mm ) ln ( miR - 223 - 5 p ) + ( nn ) ln ( miR - 582 - 3 p ) + ( oo ) ln ( miR - 122 - 3 p ) - ( pp ) ln ( miR - 589 - 5 p ) . \qquad Formula IV$$

wherein: (a)=any number from 0.1 to 4; (b)=any number from 1 to 6; (c)=any number from 13 to 25; (d)=any number from 26 to 36; (e)=any number from 3 to 12; (f)=any number from 6 to 16; (g)=any number from 9 to 20; (h)=any number from 1 to 7; (i)=any number from 0.1 to 4; (j)=any number from 25 to 35; (k)=any number from 0.25 to 2.25; (l)=any number from 11 to 22; (m)=any number from 3 to 10; (n)=any number from 0.5 to 5; (o)=any number from 1 to 6; (p)=any number from 1 to 8; (q)=any number from 1 to 8; (r)=any number from 4 to 13; (s)=any number from 1 to 7; (t)=any number from 5 to 15; (u)=any number from 1 to 10; (v)=any number from 1 to 5; (w)=any number from 1 to 6; (x)=any number from 1 to 6; (y)=any number from 8 to 18; (z)=any number from 8 to 18; (aa)=any number from 1 to 6; (bb)=any number from 4 to 13; (cc)=any number from 0.25 to 2.25; (dd)=any number from 1 to 8; (ee)=any number from 1 to 7; (ff)=any number from 3 to 10; (gg)=any number from 3 to 10; (hh)=any number from 1 to 7; (ii)=any number from 5 to 15; (jj)=any number from 0.25 to 2; (kk)=any number from 1 to 7; (ll)=any number from 1 to 5; (mm)=any number from 1 to 7; (nn)=any number from 0.1 to 3; (oo)=any number from 1 to 6; and (pp)=any number from 1 to 6; and wherein “[X]” refers to the level of “X” in the biological sample, e.g., as represented by the number of reads per sample (per million data) by a sequencer.

In some aspects, (a)=any number from 1 to 2; (b)=any number from 3 to 4; (c)=any number from 19 to 22; (d)=any number from 31 to 34; (e)=any number from 6 to 9; (f)=any number from 10 to 13; (g)=any number from 11 to 16; (h)=any number from 3 to 5; (i)=any number from 1 to 2; (j)=any number from 28 to 32; (k)=any number from 0.5 to 1.5; (l)=any number from 15 to 18; (m)=any number from 5 to 7; (n)=any number from 1.5 to 2.5; (o)=any number from 2.5 to 3.5; (p)=any number from 4 to 5; (q)=any number from 4 to 5; (r)=any number from 7 to 10; (s)=any number from 3 to 5; (t)=any number from 7 to 11; (u)=any number from 4.5 to 7; (v)=any number from 2 to 4; (w)=any number from 2.5 to 4.5; (x)=any number from 2 to 5; (y)=any number from 11 to 14; (z)=any number from 11 to 14; (aa)=any number from 2.25 to 4.25; (bb)=any number from 7 to 10; (cc)=any number from 0.75 to 2; (dd)=any number from 3 to 5.5; (ee)=any number from 2 to 5; (ff)=any number from 5 to 9; (gg)=any number from 5 to 9; (hh)=any number from 2 to 5; (ii)=any number from 7 to 12; (jj)=any number from 0.25 to 1.5; (kk)=any number from 2 to 5; (ll)=any number from 2 to 4; (mm)=any number from 3 to 5; (nn)=any number from 0.5 to 1.75; (oo)=any number from 2 to 4; and (pp)=any number from 2 to 4.

In some aspects, (a)=about 1.59; (b)=about 3.4; (c)=about 20.35; (d)=about 32.4; (e)=about 7.89; (f)=about 11.93; (g)=about 14.41; (h)=about 3.93; (i)=about 1.39; (j)=about 30.11; (k)=about 1.15; (l)=about 16.25; (m)=about 6.21; (n)=about 2.03; (o)=about 3.07; (p)=about 4.92; (q)=about 4.49; (r)=about 8.65; (s)=about 4.08; (t)=about 9.15; (u)=about 5.62; (v)=about 2.96; (w)=about 3.48; (x)=about 3.21; (y)=about 12.86; (z)=about 13.0; (aa)=about 3.22; (bb)=about 8.56; (cc)=about 1.37; (dd)=about 4.39; (ee)=about 3.18; (ff)=about 6.47; (gg)=about 6.05; (hh)=about 3.74; (ii)=about 9.45; (jj)=about 0.69; (kk)=about 3.61; (ll)=about 2.84; (mm)=about 4.07; (nn)=about 1.11; (oo)=about 2.98; and (pp)=about 3.11.

The formulas for calculating the AMR and ACR scores use sequence reads per million data with +10 added to each count to handle non-zero values. Ln represents the natural log.

In 510, server 300 determines an attribute associated with the patient based on the ACR or AMR scores. For example, the attribute may be information associated with surveillance of the patient following an organ transplant (e.g., heart transplant), whether the patient is diagnosed with ACR or AMR, or a prediction of whether the patient is at risk of being diagnosed with ACR or AMR.

With respect to diagnosis, server 300 may compare the patient-specific scores with the distinct ACR or AMR rejection score thresholds identified by the second regression model. The distinct ACR or AMR rejection score thresholds may be threshold values for determining whether the patient is diagnosed with ACR or AMR. In some aspects, for example, if the ACR or AMR scores are equal to greater than the corresponding distinct ACR or AMR rejection score thresholds, the patient may be diagnosed with ACR or AMR.

In some aspects, a subject is identified as at risk of experiencing an ACR if the ACR score is equal to or higher than about 50. In some aspects, a subject is identified as at risk of experiencing an ACR if the ACR score is equal to or higher than about 51. In some aspects, a subject is identified as at risk of experiencing an ACR if the ACR score is equal to or higher than about 52. In some aspects, a subject is identified as at risk of experiencing an ACR if the ACR score is equal to or higher than about 53. In some aspects, a subject is identified as at risk of experiencing an ACR if the ACR score is equal to or higher than about 54. In some aspects, a subject is identified as at risk of experiencing an ACR if the ACR score is equal to or higher than about 55.

In some aspects, a subject is identified as at risk of experiencing an AMR if the AMR score is equal to or higher than about 55. In some aspects, a subject is identified as at risk of experiencing an AMR if the AMR score is equal to or higher than about 56. In some aspects, a subject is identified as at risk of experiencing an AMR if the AMR score is equal to or higher than about 57. In some aspects, a subject is identified as at risk of experiencing an AMR if the AMR score is equal to or higher than about 58. In some aspects, a subject is identified as at risk of experiencing an AMR if the AMR score is equal to or higher than about 59. In some aspects, a subject is identified as at risk of experiencing an AMR if the AMR score is equal to or higher than about 60.

In some aspects, server 300 may determine that the ACR or AMR scores are normal, and continued surveillance is necessary. Alternatively, server 300 may determine that the ACR score is elevated. This indicates that the patient should start appropriate treatment, such as a corticosteroid, an anti-thymocyte globulin, tacrolimus, cyclosporine, sirolimus, everolimus, myocophenolate mofeitil, azathioprine, tocilizumab, belatacept, and any combination thereof, based on clinical severity. Alternatively, server 300 may determine that the AMR score is elevated. This may indicate that the patient should start a treatment appropriate to AMR, such as intravenous immunoglobulin, plasmapheresis, bortezomib, carfilzomib, rituximab, eculizumab, a corticosteroid, an anti-thymocyte globulin, tacrolimus, cyclosporine, sirolimus, everolimus, myocophenolate mofeitil, azathioprine, tocilizumab, belatacept, and any combination thereof. Generating the ACR and AMR scores provides an alternative to an invasive procedure (e.g., biopsy) to diagnose rejection and distinguish the subtype of rejection, allowing for precise therapy based on the test result.

In 512, server 300 identifies a heart allograft rejection in the patient based on the diagnosis of ACR or AMR. In other words, server 300 determines that the patient is rejecting the heart transplant based on the ACR or AMR diagnosis.

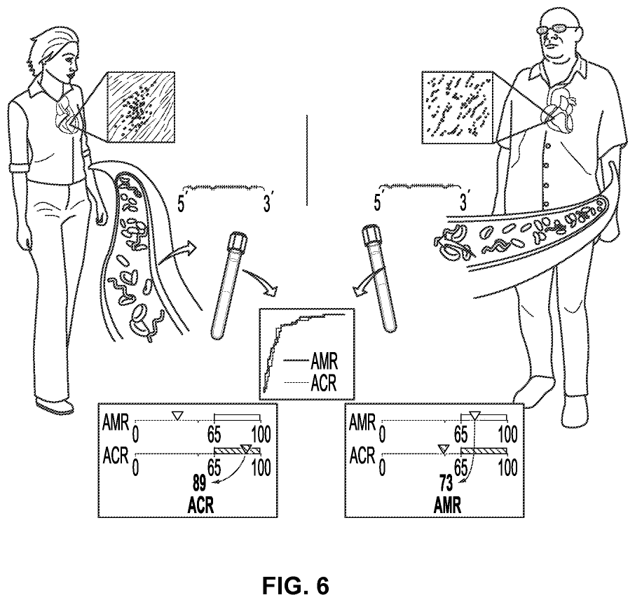

The above system and method (e.g., as shown in FIGS. 3-5) allow for diagnosing a patient with ACR or AMR based on a blood sample obtained from the patient. For example, FIG. 6 illustrates a clinical application of ACR and AMR miR scores.

As shown in FIG. 6 and using the process described in method 500 of FIG. 5, blood-based samples (e.g., whole blood, serum, and plasma) are collected in transplant patients during routine surveillance or at the time of clinically suspected allograft injury. Plasma is isolated from the whole blood, and small RNA is extracted. Small RNA-sequencing is performed to identify small RNA molecules. Sequence data is aligned to the human genome, and microRNAs are annotated using the miRBase. Expression data of individual ACR and AMR microRNAs in each patient sample is determined to allow calculation of the ACR and AMR scores, which can determine the subtype of rejection and allow for specific treatment initiation.

### IV.C.4. Computing Systems

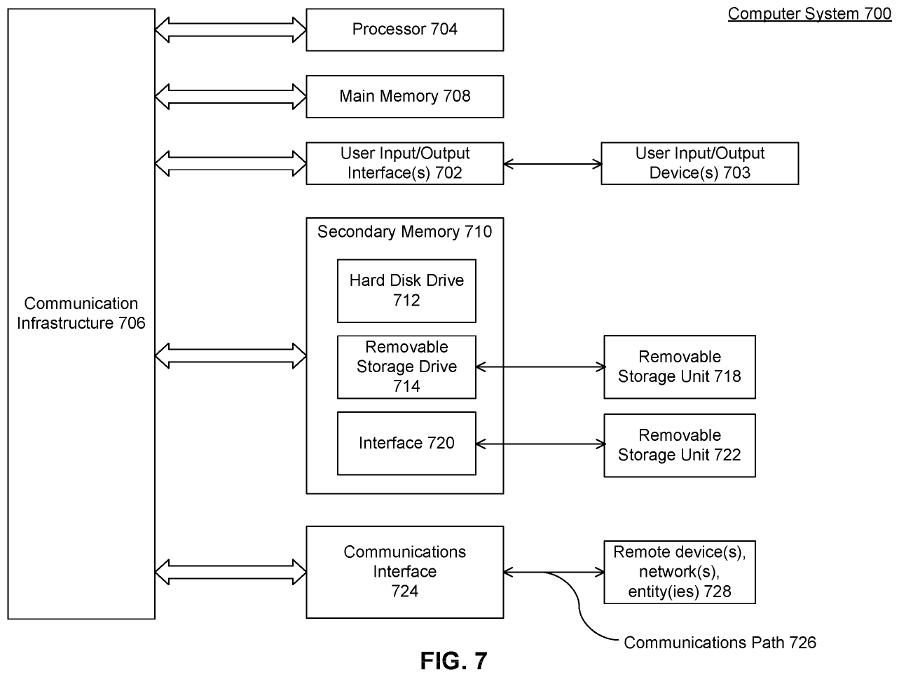

Various aspects can be implemented, for example, using one or more computer systems, such as computer system 700 shown in FIG. 7. Computer system 700 can be used, for example, to implement methods 400 of FIG. 4 and 500 of FIG. 5. Furthermore, computer system 700 can be at least part of server 300, client device 320, database 310, as shown in FIG. 3. For example, computer system 700 route communication to various applications. Computer system 700 can be any computer capable of performing the functions described herein.

Computer system 700 can be any well-known computer capable of performing the functions described herein.

Computer system 700 includes one or more processors (also called central processing units, or CPUs), such as a processor 704. Processor 704 is connected to a communication infrastructure or bus 706.

One or more processors 704 can each be a graphics processing unit (GPU). In an aspect, a GPU is a processor that is a specialized electronic circuit designed to process mathematically intensive applications. The GPU can have a parallel structure that is efficient for parallel processing of large blocks of data, such as mathematically intensive data common to computer graphics applications, images, videos, etc.

Computer system 700 also includes user input/output device(s) 703, such as monitors, keyboards, pointing devices, etc., that communicate with communication infrastructure 706 through user input/output interface(s) 702.

Computer system 700 also includes a main or primary memory 708, such as random access memory (RAM). Main memory 708 can include one or more levels of cache. Main memory 708 has stored therein control logic (i.e., computer software) and/or data.

Computer system 700 can also include one or more secondary storage devices or memory 710. Secondary memory 710 can include, for example, a hard disk drive 712 and/or a removable storage device or drive 714. Removable storage drive 714 can be a floppy disk drive, a magnetic tape drive, a compact disk drive, an optical storage device, tape backup device, and/or any other storage device/drive.

Removable storage drive 714 can interact with a removable storage unit 718. Removable storage unit 718 includes a computer usable or readable storage device having stored thereon computer software (control logic) and/or data. Removable storage unit 718 can be a floppy disk, magnetic tape, compact disk, DVD, optical storage disk, and/any other computer data storage device. Removable storage drive 714 reads from and/or writes to removable storage unit 718 in a well-known manner.

According to an exemplary aspect, secondary memory 710 can include other means, instrumentalities, or other approaches for allowing computer programs and/or other instructions and/or data to be accessed by computer system 700. Such means, instrumentalities, or other approaches can include, for example, a removable storage unit 722 and an interface 720. Examples of the removable storage unit 722 and the interface 720 can include a program cartridge and cartridge interface (such as that found in video game devices), a removable memory chip (such as an EPROM or PROM) and associated socket, a memory stick, and USB port, a memory card, and associated memory card slot, and/or any other removable storage unit and associated interface.

Computer system 700 can further include a communication or network interface 724. Communication interface 724 enables computer system 700 to communicate and interact with any combination of remote devices, remote networks, remote entities, etc. (individually and collectively referenced by reference number 728). For example, communication interface 724 can allow computer system 700 to communicate with remote devices 728 over communications path 726, which can be wired and/or wireless, and which can include any combination of LANs, WANs, the Internet, etc. Control logic and/or data can be transmitted to and from computer system 700 via communication path 726.

In an aspect, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 700, main memory 708, secondary memory 710, and removable storage units 718 and 722, as well as tangible articles of manufacture embodying any combination of the foregoing. Such control logic, when executed by one or more data processing devices (such as computer system 700), causes such data processing devices to operate as described herein.

### EXAMPLES

### Example 1: Generation of miR Panel for ACR and AMR

The following example reports the results of a multicenter cohort termed GRAfT. Using a rigorously conducted, multicenter prospective cohort study, small RNA sequencing was conducted of the circulating plasma in heart transplant patients and analyzed with paired clinical data. To validate the miR signatures, a distinct patient cohort from GRAfT and an outside collaborator were included. The aims of this analysis were to: 1) determine the miR transcriptome of heart transplant recipients; 2) distinguish miR expression in the setting of ACR and AMR; 3) develop distinct miR panels that could be used to non-invasively screen for and diagnose ACR and AMR; and 4) create individual ACR and AMR miR gene expression scores that allow for clinical interpretation and non-invasive diagnosis of acute rejection.

### Methods

### Study Design & Multicenter Discovery Cohort

The GRAfT study (NCT #02423070) was a prospective, multi-center clinical study that enrolls heart transplant recipients before transplant and follows them serially after transplant. Subjects who were 18 years of age or older were recruited and consented while on the heart transplant waitlist and monitored serially after transplantation. Patients with history of prior heart transplantation, and current pregnancy were excluded. GRAfT patients were enrolled from 2015 to 2020.

Routine post-transplant clinical care included surveillance and clinically indicated monitoring. Surveillance monitoring at pre-specified post-transplant time points included EMB for histopathology, right heart catheterization (RHC) hemodynamics, laboratory data to assess end-organ function, donor specific antibodies (DSA), cytomegalovirus (CMV) testing, as well as monitoring immunosuppressive drug levels. Clinically indicated monitoring included EMB (for cause biopsy), RHC, DSA, echocardiography, and the other tests performed when patients presented with unexplained signs or symptoms of allograft dysfunction. The study longitudinally tracked clinical data and collected blood samples coincident to both surveillance and clinically indicated monitoring.

All GRAfT patients with ACR, AMR or mixed rejection were identified during the study period. In patients with a history of both ACR and AMR, the ACR and AMR samples were analyzed separately. Patients with mixed rejection were considered with the AMR cohort, per prior publications suggesting the mixed rejection signature is most similar to AMR. Controls were selected based on freedom from clinical- or histopathologic rejection during the entirety of their clinical follow-up. The center protocols for treatment of ACR and AMR are provided.

### Histopathologic Definition of Rejection

For the purposes of this study, histopathologic rejection was characterized by ACR grade ≥2R, AMR grade ≥1 (histologic or immunologic findings) or mixed rejection. Rejection was defined based on biopsy interpretation by the individual center's pathologists to be consistent with standard clinical practice.

### Pathology Core Laboratory

Given the previously reported high discordance between pathologists in grading rejection, a blinded core lab with two expert cardiac pathologists (GB and CM) reviewed a subset of all histopathologic slides within GRAfT. The core lab read was compared to the center read, and the analysis was repeated to assess miR performance against the blinded core lab read.

### Independent Validation Cohorts

Using distinct patient samples from GRAfT, an independent validation was performed of the ACR and AMR miR signatures. In addition to permit external validation, a similar prospective, single-center study that was conducted at Stanford University (NCT #01985412) between 2011 and 2018 was also used. Stanford University patients were recruited shortly after heart transplantation under similar exclusion criteria and blood was banked using similar methods as GRAfT. The methods used herein were able to identify cases with ACR and controls with no history of rejection after transplant. The ACR miR signatures were tested for performance in the Stanford cohort. The prevalence of AMR in the Stanford cohort was low; thus, this validation focused on ACR alone.

### Sample Collection, MicroRNA Extraction and Sequencing

Blood samples were collected during surveillance or clinically indicated testing. For patients undergoing EMB, the blood samples were collected before the procedure. Venous blood was collected into Streck Cell-Free DNA tubes and processed to isolate plasma, which was stored at −80° C. Total RNA was extracted using the MagMAX mirVana isolation kit (Thermo Fisher Scientific, Gaithersburg MD) and after elution, library preparation was performed using the NEXTFLEX Small RNA-Seq Kit v3 (PerkinElmer Inc, Shelton CT). Sequencing was performed on the NextSeq 500 (Illumina Inc., San Diego CA) using 50 bp single-end reads, generating ˜11 million reads per sample.

### Bioinformatic Analysis of Sequencing Data

Small RNA sequence data was analyzed using the Extracellular RNA Communication Consortium (ERCC) RNA pipeline (extracellular RNA processing tool, exceRpt). In brief, sequence FASTQ files were processed to remove 3′ adapter barcode sequences, random-barcode sequences, UniVec contaminants, and reads <15 bp. High-quality filtered sequences were then endogenously aligned using miRbase v22, GENCODE v24 for the reference human genome 38, and gtRNAdb v16. Samples with insufficient miR reads (<1,000,000 reads per sample) of good quality were excluded from the analysis. To ensure there was no batch effect across small RNA sequencing runs, a principal component analysis was performed using Caenorhabditis elegans sample spike-ins and a pooled plasma control from unrelated human donors (data not presented). Outlier samples were then excluded from further bioinformatic/biostatistical analysis. The laboratory technicians and bioinformatics team were blinded to the biopsy results.

Gene targets of miRs implicated in ACR and AMR were identified using miRTarBase v8.0. Only experimentally validated mRNA targets through reporter assays, western blot, or microarray experiments with overexpression or knockdown of miRs were included in the analysis. Network analysis and data visualization were performed with MIENTURNET. To distinguish biological pathways potentially regulated by the differentially expressed miRs, the Reactome database was searched.

### Biostatistical Analysis

Small RNA sequencing led to identification of ˜1,900 expressed miRs in plasma. The average expression across all samples was 475±124 miRs. Lowly expressed miRs, with less than 100 average mapped reads across all samples, were filtered out. In addition, miR 486-5p and miR-451a which are red blood cell derived miRs were removed for the analysis. The remaining 286 miRs were included in the analysis. A two-tier analysis was conducted to identify the significant miRs in discriminating cases (ACR or AMR) from controls. For the first-tier analysis, the 286 miRs were screened to identify differentially expressed miRs in ACR and AMR while adjusting for clinical covariates (age, sex, race, body-mass index) using DESeq2. Blood group was adjusted for in a subset of patients with minimal change in miR profile, but the data was missing in other cases, and in order to maximize sample size, the data presented is adjusted for age, sex, race, and body-mass index.

For the second-tier analysis, a logistic regression models were fitted with a LASSO penalty using the miRs with an unadjusted p-value <0.10 identified from the differential gene expression analysis. Within the LASSO analysis, the normalized miR counts were log-transformed to approximate normality and the log counts for each miR were standardized to have a mean of zero and variance of one. The tuning parameter of the LASSO penalty was chosen by 10-fold cross-validation to minimize model deviance.

Receiver operating characteristic (ROC) curves were generated and the area under the curves (AUC) were calculated to assess the performance of the LASSO-selected miRs in the independent validation cohort from GRAfT for ACR and AMR, and Stanford University for ACR. To assess the predictive ability of the miRs, AUC statistics were generated with 10-fold cross-validation.

Finally, a logistic regression model was fitted for ACR and AMR using the counts per million data for the miRs selected by the logistic-LASSO regression. The ordinary logistic regression estimates are prone to bias due to our low sample size and the large observed difference in expression of some miRs in cases and controls (which causes the likelihood function to be flat near the maximum), particularly for AMR. A bias reduced maximum likelihood estimator was used for the final logistic regression model parameters. ROC curves were generated using the sequenced samples from the GRAfT cohort. Youden's index was used to identify a threshold to maximize test performance. Test performance characteristics are presented including sensitivity, specificity, negative predictive value (NPV) and positive predictive value (PPV). These distinct ACR and AMR rejection scores are based on the miR expression data for each patient sample.

### Results

### Patient Characteristics

During the study period a total 116 heart transplant patients from GRAfT had completed one-year clinical follow-up and had plasma samples available for inclusion in the study. The median patient age was 52 years (IQR: 42-59), 35% of patients were female, and 45% were Black. The most common cause of HF was non-ischemic cardiomyopathy (63%) and 67% of patients were bridged to transplant with a durable left ventricular assist device (LVAD, Table 1). The median follow-up time after transplantation was 3.73 patient-years (IQR: 3.23-4.15).

TABLE 1
GRAfT patient characteristics at time of transplant with and without acute rejection.

| | | | | | | | | | p-value |
|---|---|---|---|---|---|---|---|---|---|
| | GRAfT | | Control | | ACR | | AMR | | for ACR |
| Characteristic | (N = 116) | | (N = 85) | | (N = 20) | | (N = 14) | | vs AMR |
| Age, median (IQR) | 51.5 | (42.0, 59.0) | 53.0 | (44.0, 60.0) | 49.5 | (39.0, 55.3) | 49.5 | (35.3, 52.8) | 0.12 |
| Sex | | | | | | | | | 0.84 |
| Female | 41 | (35.3%) | 31 | (36.5%) | 7 | (35.0%) | 4 | (28.6%) | |
| Male | 75 | (64.7%) | 54 | (63.5%) | 213 | (65.0%) | 10 | (71.4%) | |
| Race | | | | | | | | | 0.05 |
| Black | 52 | (44.8%) | 40 | (47.1%) | 9 | (45.0%) | 12 | (85.7%) | |
| White | 58 | (50%) | 39 | (45.9%) | 11 | (55.0%) | 2 | (14.3%) | |
| Other | 6 | (5.2%) | 6 | (7.1%) | 0 | (0.0%) | 0 | (0%) | |
| Ethnicity | | | | | | | | | 0.67 |
| Hispanic or | 8 | (6.9%) | 5 | (5.9%) | 2 | (10%) | 1 | (7.1%) | |
| Latino | | | | | | | | | |
| Not Hispanic | 108 | (93.1%) | 80 | (94.1%) | 18 | (90%) | 12 | (92.9%) | |
| or Latino | | | | | | | | | |
| Body-mass index, | 27.9 | (24.8, 32.1) | 27.6 | (24.0, 32.1) | 27.4 | (25.2, 30.8) | 30.6 | (28.4, 33.1) | 0.16 |
| kg/m² | | | | | | | | | |
| Cardiomyopathy | | | | | | | | | 0.36 |
| Type | | | | | | | | | |
| Ischemic | 23 | (20.2%) | 15 | (17.7%) | 4 | (22.2%) | 4 | (28.6%) | |
| Non-ischemic | 72 | (63.2%) | 56 | (65.9%) | 11 | (61.1%) | 8 | (43.2%) | |
| Other | 19 | (16.7%) | 14 | (16.4%) | 2 | (16.8%) | 2 | (14.3%) | |
| UNOS Status at | | | | | | | | | |
| Transplant | | | | | | | | | |
| 1A | 75 | (72.1%) | 51 | (68.9%) | 17 | (85%) | 10 | (76.9%) | 0.42 |
| 1B | 26 | (25%) | 21 | (28.4%) | 3 | (15%) | 2 | (15.4%) | |
| 2 | 3 | (2.9%) | 2 | (2.7%) | 0 | (3%) | 1 | (7.7%) | |
| Bridged to | 70 | (67.3%) | 52 | (67.5%) | 9 | (50%) | 11 | (91.7%) | 0.06 |
| Transplant with | | | | | | | | | |
| LVAD | | | | | | | | | |
| Medical Co- | | | | | | | | | |
| morbidities | | | | | | | | | |
| HTN | 79 | (70.5%) | 62 | (75.6%) | 10 | (52.6%) | 9 | (64.3%) | 0.12 |
| HLP | 42 | (37.8%) | 30 | (36.6%) | 6 | (33.3%) | 8 | (57.1%) | 0.30 |
| DM | 34 | (30.6%) | 21 | (25.9%) | 7 | (36.8%) | 7 | (50%) | 0.16 |
| Prior Smoking | 45 | (38.8%) | 35 | (41.2%) | 4 | (20%) | 8 | (57.1%) | 0.07 |
| Creatinine, mg/dl | 1.20 | (1.00, 1.46) | 1.20 | (1.00, 1.47) | 1.00 | (0.86, 1.40) | 1.40 | (1.20, 2.14) | 0.03 |

An incidence of AR of 15.8% was previously reported within GRAfT (Agbor-Enoh & Shah et al., Circulation 143:1184 (2021). Only an EMB with concomitant miR sequencing was included in this analysis. Patients were divided into a discovery and validation cohort (FIG. 1A). In the discovery miR analysis, 20 episodes of ACR (all grade 2R, no 3R episodes), 14 episodes of AMR (AMR≥2, n=6; AMR 1, n=8), and 2 episodes of mixed ACR/AMR were included. There were 3 patients with separate instances of ACR and AMIR on different EMB and each episode of rejection was analyzed separately. The median time to ACR was 146 days (50-496) and AMIR 55 days (21-171).

Median age for patients with ACR or AMR was 49.5 years, younger than controls without rejection 53.0 years (Table 1). Black patients had a higher incidence of AMR (85.7%) compared to White patients (14.3%, p=0.05), and bridging to transplant with an LVAD was associated with a higher incidence of AMR compared to non-bridged patients (91.7% v. 8.3%, p=0.06).

### MicroRNA Sequencing

A total of 405 plasma samples underwent small RNA sequencing between GRAfT and Stanford University. A total of 10.9±3.1 million small RNA reads were generated per sample. This includes miR, piwi RNA, small nucleolar RNA, transfer RNA and unmapped RNA species. After filtering non-miR reads, each sample generated approximately 5.6±2.7 million miR reads (Table 2). There were, on average, 475±124 individual miRs expressed per sample, with 286±45 miRs detected at a depth of ≥100 read per sample. The top 20 miRs accounted for 75% of the total miR transcriptome in heart transplant patients, with miR-451a and miR-486-5p being the most abundant which were removed from the analysis due to their predominate origin from red blood cells.

TABLE 2
Small Molecular RNA Sequencing Characteristics

| | | Standard |
|---|---|---|
| Parameter | Average | Deviation |
| Total reads per sample (million) | 10.9 | 3.1 |
| Total transcriptome reads per sample (million) | 5.9 | 2.7 |
| Total microRNA reads per sample (million) | 5.6 | 2.7 |
| # MicroRNAs expressed per sample (absolute | 475 | 124 |
| mapped reads) | | |
| # number of miRs detected at a depth of 10 | 410 | 96 |
| reads or greater (≥10 reads) per sample. | | |
| # number of miRs detected at a depth of 100 | 286 | 45 |
| reads or greater (≥100 reads) per sample. | | |

Differential miR Analysis in Rejection & Target Pathways for Dysregulated miRs

Using the GRAfT discovery cohort, a differential gene expression analysis was performed comparing the miR profile of control patients (without rejection) and patients with ACR. When comparing these patient populations, 12 miRs were identified with a p-value <0.05 (FIG. 1B; Table 3). Target prediction analysis was performed with the differentially expressed ACR miRs using miRTarBase. Only previously published, experimentally validated miR-mRNA target interactions were included to ensure a high level of biologic relevance. A total of 294 genes were targeted by the ACR miRs and many of these genes are implicated in mTOR signaling, T-cell differentiation, transforming growth factor-β (TGF-β) signaling, and T-cell receptor (TCR) signaling.

TABLE 3
Differentially Expressed MicroRNAs in ACR and AMR

| ACR | Mean | log₂ Fold | Standard | | |
|---|---|---|---|---|---|
| MicroRNAs | Expression | Change | Error | Statistic | P value |
| miR-223-3p | 45940 | −0.93 | 0.25 | −3.70 | 0.000 |
| miR-361-3p | 1049 | 0.42 | 0.14 | 2.93 | 0.003 |
| miR-3615 | 1364 | −0.43 | 0.16 | −2.75 | 0.006 |
| miR-24-3p | 37695 | −0.39 | 0.16 | −2.46 | 0.014 |
| miR-182-5p | 790 | 0.72 | 0.30 | 2.41 | 0.016 |
| miR-374a-5p | 883 | 0.42 | 0.18 | 2.28 | 0.023 |
| miR-23a-3p | 57985 | −0.39 | 0.17 | −2.27 | 0.023 |
| miR-30e-5p | 78017 | −0.18 | 0.09 | −2.09 | 0.037 |
| miR-582-3p | 340 | −0.95 | 0.46 | −2.04 | 0.041 |
| miR-130b-3p | 442 | −0.41 | 0.20 | −2.03 | 0.042 |
| miR-326 | 92 | −1.05 | 0.52 | −2.03 | 0.043 |
| miR-1299 | 100 | −2.25 | 1.14 | −1.98 | 0.048 |
| AMR | Mean | log₂ Fold | Standard | | |
| MicroRNAs | Expression | Change | Error | Statistic | P value |
| miR-23a-3p | 58794 | −0.80 | 0.20 | −3.94 | 0.000 |
| miR-145-5p | 605 | −1.30 | 0.36 | −3.62 | 0.000 |
| miR-1249-3p | 411 | −1.31 | 0.39 | −3.32 | 0.001 |
| miR-27a-3p | 28287 | −0.74 | 0.22 | −3.32 | 0.001 |
| miR-215-5p | 815 | −1.76 | 0.54 | −3.23 | 0.001 |
| miR-145-3p | 1324 | −0.90 | 0.30 | −3.04 | 0.002 |
| miR-10b-5p | 24845 | −0.82 | 0.27 | −2.98 | 0.003 |
| miR-582-3p | 338 | −1.57 | 0.55 | −2.86 | 0.004 |
| let-7b-3p | 1349 | −0.55 | 0.20 | −2.80 | 0.005 |
| miR-142-3p | 1193 | 0.78 | 0.28 | 2.78 | 0.005 |
| miR-450b-5p | 1643 | −0.85 | 0.31 | −2.76 | 0.006 |
| miR-140-5p | 506 | 0.84 | 0.31 | 2.71 | 0.007 |
| miR-374a-5p | 906 | 0.57 | 0.22 | 2.57 | 0.010 |
| miR-17-5p | 1609 | 0.61 | 0.24 | 2.56 | 0.010 |
| miR-143-3p | 51349 | −0.90 | 0.35 | −2.55 | 0.011 |
| miR-130b-3p | 463 | −0.55 | 0.23 | −2.37 | 0.018 |
| miR-1-3p | 3288 | −0.93 | 0.40 | −2.34 | 0.019 |
| miR-542-3p | 738 | −0.80 | 0.35 | −2.28 | 0.022 |
| miR-484 | 9962 | 0.51 | 0.23 | 2.22 | 0.027 |
| miR-345-5p | 1217 | −0.67 | 0.31 | −2.16 | 0.031 |
| miR-125a-5p | 9648 | −0.57 | 0.27 | −2.14 | 0.032 |
| miR-338-5p | 879 | −0.92 | 0.43 | −2.11 | 0.035 |
| miR-769-5p | 351 | −0.97 | 0.46 | −2.10 | 0.036 |
| miR-193a-5p | 3018 | −0.67 | 0.32 | −2.08 | 0.038 |
| miR-454-3p | 858 | 0.63 | 0.31 | 2.06 | 0.040 |
| miR-223-5p | 2258 | −0.84 | 0.43 | −1.97 | 0.049 |
| let-7d-3p | 24761 | −0.26 | 0.13 | −1.97 | 0.049 |

Similarly, GRAfT patients with AMR were compared to control patients without rejection, and 27 differentially expressed miRs were identified with a p-value <0.05 (FIG. 1C, Table 3). A total of 478 genes were targeted by the AMR miRs and many of these genes are involved in pathways affecting interleukin signaling, downstream T-cell receptor signaling, tumor necrosis factor (TNF) signaling and toll-like receptor (TLR) cascades.

Using LASSO regression with internal cross-validation, a panel of 11 miRs was identified that accurately discriminated ACR from controls. Similarly, 15 miRs were identified that accurately discriminated AMR from controls. The internal correlation between these ACR and AMR miRs was low (data not presented). Only 2 miRs were common to both the ACR and AMR panels (miR-130b-3p and miR-374a-5p).

### ACR and AMR miR Expression Before and After Rejection

To understand whether miRs could serve as biomarkers of future rejection and/or predict response to therapy, the patterns of miR expression, before, during and after a rejection episode were examined. Using the miR panels identified by LASSO regression, the majority of differentially expressed miRs in ACR are up regulated at the time of allograft rejection and return to control levels once the rejection episode is treated (miR374a-5p, FIG. 1D; miR-345-5p, FIG. 1F). There were however certain miRs with altered expression prior to ACR onset compared to controls (miR-182-5p, FIG. 1E; miR-130b-3p, FIG. 1G).

In AMR, a number of miRs are dysregulated prior to the clinical diagnosis of rejection (miR-23a-3p, FIG. 1H; miR-340-5p, FIG. 1J). After treatment of AMR rejection certain miRs return to normal levels (miR-142-3p, FIG. 1I; miR-185-3p, FIG. 1K), while others remain persistently dysregulated (miR-23a-3p, FIG. 1H; miR-340-5p, FIG. 1J).

### miR Performance for Rejection Diagnosis in Validation Cohorts

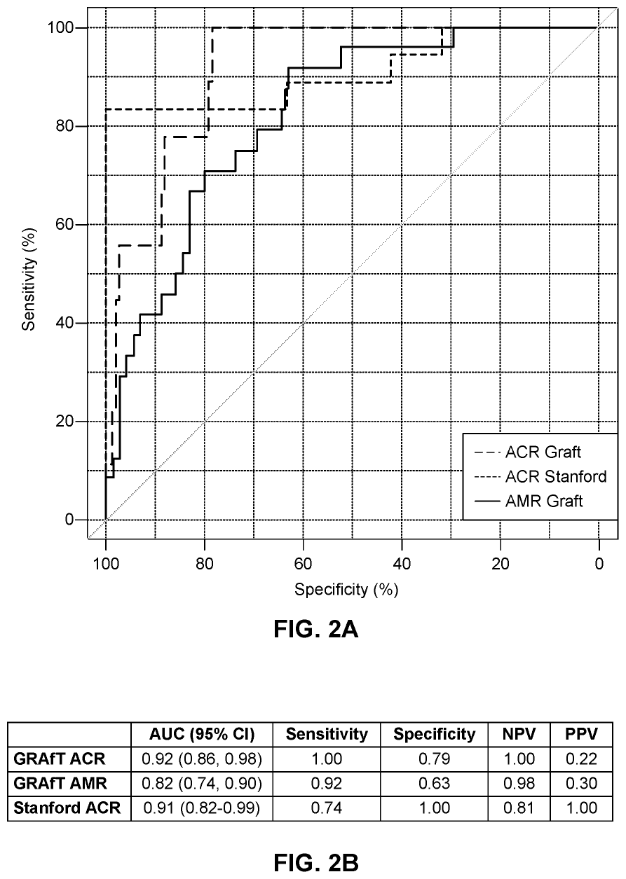

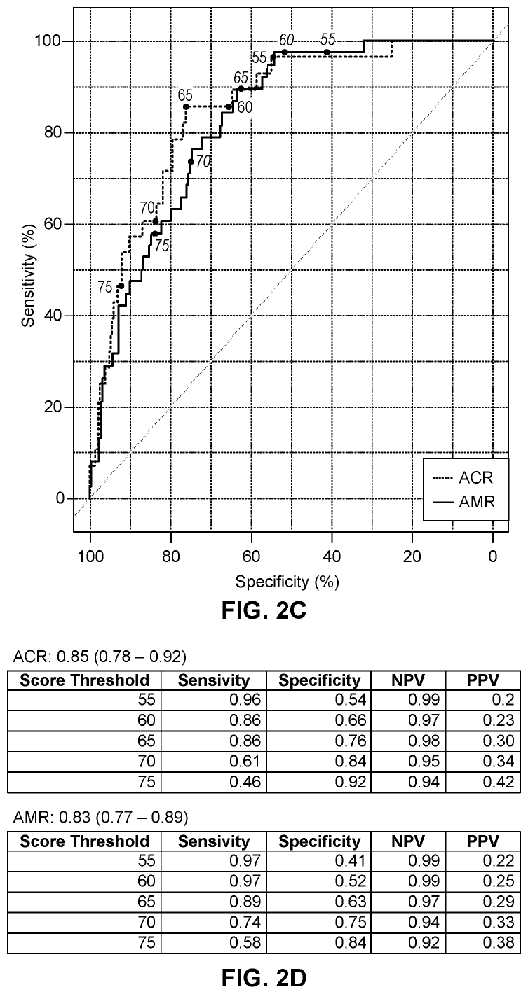

To discriminate performance of our selected miR panels to non-invasively diagnosis ACR and AMR, validation was performed in an independent cohort of GRAfT patient samples and calculated AUC statistics. The performance characteristics for ACR and AMR had an ACR AUC of 0.92 (95% CI: 0.86-0.98) and for AMR an AUC of 0.82 (95% CI: 0.74-0.90, FIGS. 2A-2B). This leads to a sensitivity ranging between 92%-100%, and specificity ranging between 63-79%.

To perform additional external validation, the miR transcriptome in a transplant cohort from Stanford University (n=41) was sequenced. Stanford University patients tended to be older than GRAfT (57.5 years compared to 51.5 years), had a lower proportion of female sex patients (19.5% compared 35.3%), Black race patients (12.2% compared to 44.8%) and bridging with an LVAD (43.9% compared to 67.3%). Patients in the Stanford cohort had more renal dysfunction prior to transplant. Other patient characteristics were similar between the cohorts. The AUC of the ACR miR panel in the Stanford cohort was 0.91 (95% CI: 0.82-0.99, FIGS. 2A-2B), leading to a negative predictive value of 81% and positive predictive value of 100%.

### Core Pathology Lab Analysis

Two blinded cardiac pathologists (GP and CM) reviewed a subset of EMB histopathologic slides from GRAfT patients to verify the presence of ACR and AMR. Of the 263 biopsies included in the analysis, 95 (36%) were reviewed by the blind core lab. Overall concordance amongst pathologist for the histopathologic interpretation of EMB was 63%, for biopsies without rejection by the center read concordance was 79%, and for rejection samples concordance was only 28%. The lowest concordance was seen in AMR overall (Table 4).

TABLE 4
Comparison of Center and Blinded Core Lab Histopathologic
Interpretation of Heart Biopsy Slides for ACR and AMR

| | | CENTER READ | |
|---|---|---|---|
| | ACR Grade | 2R | 1R or 0 |
| BLINDED CORE LAB | 2R | 4 | 11 |
| | 1R or 0 | 10 | 70 |
| | | Center Read | |
| | AMR Grade | pAMR1, 2 or 3 | pAMR0 |
| BLINDED CORE LAB | pAMR1, 2 or 3 | 3 | 4 |
| | pAMR0 | 13 | 75 |

Using the ACR and AMR miR panels, performance at detection of acute rejection based on the blinded core lab read was assessed. The selected ACR miR panel led to an AUC of 0.85 (95% CI: 0.75-0.95). The AMR miR panel led to an AUC of 0.96 (95% CI: 0.87-1.00).

### Development of Circulating MicroRNA Clinical Rejection Scores for ACR and AMR

Using logistic regression, distinct ACR and AMR clinical rejection scores were developed, which were scaled from 0-100. In the entire GRAfT patient cohort, an individual miR ACR and AMR score was calculated for each biopsy time point:

$$ACR SCORE = 251.89 - 28.9 * ln ( miR - 30 e - 5 p ) - 0.19 * ln ( let - 7 g - 5 p ) - 5.46 * ln ( miR - 223 - 3 p ) + 4.77 * ln ( miR - 3615 ) + 6.41 * ln ( miR - 374 a - 5 p ) + 4.41 * ln ( miR - 182 - 5 p ) - 2.2 * ln ( miR - 345 - 5 p ) + 27.69 * ln ( miR - 361 - 3 p ) - 3.05 * ln ( miR - 130 b - 3 p ) - 6.17 * ln ( miR - 1299 ) 7. 71 * ln ( miR - 376 c - 3 p ) - 10.63 * ln ( miR - 326 ) \qquad Formula I$$

$$AMR SCORE = 222.41 - 25.44 * ln ( miR - 23 a - 3 p ) - 9.33 * ln ( miR - 484 ) - 3.39 * ln ( miR - 340 - 5 p ) + 5.82 * ln ( miR - 193 a - 5 p ) - 8.24 * ln ( miR - 215 - 5 p ) + 8.62 * ln ( miR - 142 - 3 p ) - 2.75 * ln ( miR - 374 a - 5 p ) + 1.43 * ln ( miR - 1307 ) + 7.95 * ln ( miR - 185 - 3 p ) + 9.69 * ln ( miR - 4433 b - 3 p ) + 5.47 * ln ( miR - 130 b - 3 p ) + 0.6 * ln ( miR - 331 - 5 p ) + 2.05 * ln ( miR - 140 - 5 p ) + 2.24 * ln ( miR - 223 - 5 p ) + 1.4 * ln ( miR - 582 - 3 p ) + 0.87 * ln ( miR - 122 - 3 p ) + 1.69 * ln ( miR - 589 - 5 p ) \qquad Formula III$$

Using reads per million data with +10 added to each count to handle non-zero values. Ln represents the natural log.

ROC curves were generated, and Youden's Index was used to identify the threshold of the score to maximize the AUC and test performance characteristics (FIGS. 2C-2D). The point that maximized sensitivity and specificity was 65. The ACR score threshold of 65 led to an AUC of 0.85 (95% CI: 0.78-0.92), the associated test characteristics were a sensitivity of 86%, specificity of 76%, NPV of 98% and PPV of 30%. For AMR the AUC was 0.83 (95% CI: 0.77-0.89). An AMR score threshold of 65 led to a sensitivity of 89%, specificity of 63%, NPV of 97% and PPV of 29%. The score threshold can be increased or decreased to maximize test sensitivity and specificity as demonstrated in FIGS. 2C-2D.

### CONCLUSION

The analysis according to aspects described herein identified a distinct set of miRs that can be used to non-invasively diagnose ACR and AMR from a peripheral blood sample with excellent test performance characteristics. These miR panels were validated with additional patient samples from GRAfT and an external validation cohort. Since there are distinct ACR and AMR scores; it allows for a non-invasive blood test to be used to not only screen for rejection, but also diagnose the subtype of rejection present. This type of testing that distinguishes ACR from AMR from no rejection, permits the initiation of targeted therapy while awaiting additional diagnostic testing.

The transplant community has spent the greater part of the past 3 decades searching for reliable, non-invasive biomarkers to detect acute allograft rejection. Current biomarkers include gene expression profiling (GEP), soluble protein biomarkers, donor-derived cell-free DNA (dd-cfDNA), and T-cell immune function assays. Commercially-available gene-expression profiling (GEP) involves the measurement of 11 mRNA transcripts implicated in immune system function. Widescale implementation and reliance on GEP testing has, however, been limited by its poor PPV (˜10%) and inability to detect AMR. More recently, through sequencing of a panel of highly informative single-nucleotide polymorphisms (SNPs) in circulating, cell-free DNA; SNP mismatches in donor and recipient DNA can be used to quantify the donor-derived portion of cell-free DNA (dd-cfDNA). The percent of plasma dd-cfDNA has been shown to correlate with the presence and severity of allograft rejection, and is a biomarker for allograft injury. Prior work has validated dd-cfDNA as a highly sensitive non-invasive biomarker of both ACR and AMR. However a critical limitation is that dd-cfDNA in its current application cannot accurately discriminate between ACR and AMR and an EMB is still required.

In the aforementioned tests according to aspects described herein, unique miR subsets were identified that discriminate the presence of ACR and AMR from patients without rejection with an excellent NPV (˜98%). These ACR and AMR miR scores can be used as part of a post-transplant non-invasive surveillance strategy. Since the miR scores are unique to ACR or AMR, if elevated, clinicians can start targeted therapy (e.g., steroids and/or thymoglobulin for ACR v. plasmapheresis and intravenous immunoglobulin for AMR), while awaiting results from other diagnostic testing: DSA, echocardiogram, EMB, and/or dd-cfDNA.

Based on the teachings contained in this disclosure, it will be apparent to persons skilled in the relevant art(s) how to make and use aspects of this disclosure using data processing devices, computer systems, and/or computer architectures other than that shown in FIG. 11. In particular, aspects can operate with software, hardware, and/or operating system implementations other than those described herein.

It is to be appreciated that the Detailed Description section, and not any other section, is intended to be used to interpret the claims. Other sections can set forth one or more but not all exemplary aspects as contemplated by the inventor(s), and thus, are not intended to limit this disclosure or the appended claims in any way.

While this disclosure describes exemplary aspects for exemplary fields and applications, it should be understood that the disclosure is not limited thereto. Other aspects and modifications thereto are possible and are within the scope and spirit of this disclosure. For example, and without limiting the generality of this paragraph, aspects are not limited to the software, hardware, firmware, and/or entities illustrated in the figures and/or described herein. Further, aspects (whether or not explicitly described herein) have significant utility to fields and applications beyond the examples described herein.

Aspects have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative aspects can perform functional blocks, steps, operations, methods, etc., using orderings different than those described herein.

References herein to “one aspect,” “an aspect,” “an example aspect,” or similar phrases indicate that the aspect described can include a particular feature, structure, or characteristic, but every aspect can not necessarily include the particular feature, structure, or characteristic. Moreover, such phrases are not necessarily referring to the same aspect. Further, when a particular feature, structure, or characteristic is described in connection with an aspect, it would be within the knowledge of persons skilled in the relevant art(s) to incorporate such feature, structure, or characteristic into other aspects whether or not explicitly mentioned or described herein. Additionally, some aspects can be described using the expression “coupled” and “connected” along with their derivatives. These terms are not necessarily intended as synonyms for each other. For example, some aspects can be described using the terms “connected” and/or “coupled” to indicate that two or more elements are in direct physical or electrical contact with each other. The term “coupled,” however, can also mean that two or more elements are not in direct contact with each other but yet still co-operate or interact with each other.

The breadth and scope of this disclosure should not be limited by any of the above-described exemplary aspects but should be defined only in accordance with the following claims and their equivalents.

1. A microfluidic array comprising one or more RNA-hybridization probes, wherein at least one of the RNA-hybridization probes hybridizes to an miR selected from the group consisting of miR-223-3p, miR-361-3p, miR-3615, miR-24-3p, miR-182-5p, miR-374a-5p, miR-23a-3p, miR-30e-5p, miR-582-3p, miR-130b-3p, miR-326 92, miR-1299, miR-23a-3p, miR-145-5p, miR-1249-3p, miR-27a-3p, miR-215-5p, miR-145-3p, miR-10b-5p, miR-582-3p, let-7b-3p, miR-142-3p, miR-450b-5p, miR-140-5p, miR-374a-5p, miR-17-5p, miR-143-3p, miR-130b-3p, miR-1-3p, miR-542-3p, miR-484, miR-345-5p, miR-125a-5p, miR-338-5p, miR-769-5p, miR-193a-5p, miR-454-3p, miR-223-5p, and let-7d-3p.

2. The microfluidic array of claim 1, wherein at least one of the RNA-hybridization probes hybridizes to an miR selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326, and any combination thereof.

3. The microfluidic array of claim 2, comprising an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, an RNA-hybridization probe that hybridizes to miR-326.

4. The microfluidic array of claim 1, wherein at least one of the RNA-hybridization probes hybridizes to an miR selected from the group consisting of miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, miR-589-5p, and any combination thereof.

5. The microfluidic array of claim 4, comprising an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, and an RNA-hybridization probe that hybridizes to miR-589-5p.

6. The microfluidic array of claim 1, wherein the microfluidic array comprises a fluidic chip comprising one or more RNA-hybridization probes, wherein at least one of the RNA-hybridization probes hybridizes to an miR selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326, and any combination thereof.

7. The microfluidic array of claim 6, wherein the fluidic chip comprises an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, an RNA-hybridization probe that hybridizes to miR-326.

8. The microfluidic array of claim 1, wherein the microfluidic array comprises a fluidic chip comprising one or more RNA-hybridization probes, wherein at least one of the RNA-hybridization probes hybridizes to an miR selected from the group consisting of miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, miR-589-5p, and any combination thereof.

9. The microfluidic array of claim 8, wherein the fluidic chip comprises an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, an RNA-hybridization probe that hybridizes to and miR-589-5p.

10. (canceled)

11. A panel of RNA-hybridization probes that hybridize to one or more miR selected from the group consisting of miR-223-3p, miR-361-3p, miR-3615, miR-24-3p, miR-182-5p, miR-374a-5p, miR-23a-3p, miR-30e-5p, miR-582-3p, miR-130b-3p, miR-326 92, miR-1299, miR-23a-3p, miR-145-5p, miR-1249-3p, miR-27a-3p, miR-215-5p, miR-145-3p, miR-10b-5p, miR-582-3p, let-7b-3p, miR-142-3p, miR-450b-5p, miR-140-5p, miR-374a-5p, miR-17-5p, miR-143-3p, miR-130b-3p, miR-1-3p, miR-542-3p, miR-484, miR-345-5p, miR-125a-5p, miR-338-5p, miR-769-5p, miR-193a-5p, miR-454-3p, miR-223-5p, let-7d-3p, and any combination thereof for use in identifying a human subject afflicted with or at risk of developing an acute heart allograft rejection following a heart transplant.

12. The panel of RNA-hybridization probes of claim 11, wherein the acute heart allograft rejection comprises ACR, AMR, or a combination thereof.

13. The panel of RNA-hybridization probes of claim 11, wherein the RNA-hybridization probes hybridize one or more miR selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326, and any combination thereof for use in identifying a human subject afflicted with or at risk of developing ACR following a heart transplant.

14. (canceled)

15. The panel of RNA-hybridization probes of claim 13, comprising an RNA-hybridization probe that hybridizes to miR-30e-5p, an RNA-hybridization probe that hybridizes to let-7g-5p, an RNA-hybridization probe that hybridizes to miR-223-3p, an RNA-hybridization probe that hybridizes to miR-3615, an RNA-hybridization probe that hybridizes to miR-374a-5p, an RNA-hybridization probe that hybridizes to miR-182-5p, an RNA-hybridization probe that hybridizes to miR-345-5p, an RNA-hybridization probe that hybridizes to miR-361-3p, an RNA-hybridization probe that hybridizes to miR-130b-3p, an RNA-hybridization probe that hybridizes to miR-1299, an RNA-hybridization probe that hybridizes to miR-376c-3p, and an RNA-hybridization probe that hybridizes to miR-326.

16. (canceled)

17. The panel of RNA-hybridization probes of claim 11, wherein the RNA-hybridization probes hybridize one or more miR selected from the group consisting of miR-23a-3p, miR-484, miR-340-5p, miR-193a-5p, miR-215-5p, miR-142-3p, miR-374a-5p, miR-1307, miR-185-3p, miR-4433b-3p, miR-130b-3p, miR-331-5p, miR-140-5p, miR-223-5p, miR-582-3p, miR-122-3p, miR-589-5p, and any combination thereof for use in identifying a human subject afflicted with or at risk of developing AMR following a heart transplant.

18. (canceled)

19. The panel of RNA-hybridization probes of claim 17, comprising an RNA-hybridization probe that hybridizes to miR-23a-3p, an RNA-hybridization probe that hybridizes to miR-484, an RNA-hybridization probe that hybridizes to miR-340-5p, an RNA-hybridization probe that hybridizes to miR-193a-5p, an RNA-hybridization probe that hybridizes to miR-215-5p, an RNA-hybridization probe that hybridizes to miR-142-3p, an RNA-hybridization probe that hybridizes to miR-374a-5p, an RNA-hybridization probe that hybridizes to miR-1307, an RNA-hybridization probe that hybridizes to miR-185-3p, an RNA-hybridization probe that hybridizes to miR-4433b-3p, an RNA-hybridization probe that hybridizes to miR-130b-3p, an RNA-hybridization probe that hybridizes to miR-331-5p, an RNA-hybridization probe that hybridizes to miR-140-5p, an RNA-hybridization probe that hybridizes to miR-223-5p, an RNA-hybridization probe that hybridizes to miR-582-3p, an RNA-hybridization probe that hybridizes to miR-122-3p, an RNA-hybridization probe that hybridizes to and miR-589-5p.

20. (canceled)

21. A method of identifying a human subject experiencing or at risk of experiencing an acute heart allograft rejection comprising an acute cellular rejection (ACR) following a heart transplant comprising:
(i) obtaining a biological sample from the human subject, wherein the biological sample comprises RNA,
(ii) contacting the biological sample comprising the RNA with the panel of claim 13; and
(iii) determining an ACR signature score according to the following formula:
$$ACR signature score = 251.89 - ( a ) * ln [ miR - 30 e - 5 p ] - ( b ) * ln [ let - 7 g - 5 p ] - ( c ) * ln [ miR - 223 - 3 p ] + ( d ) * ln [ miR - 3615 ] + ( e ) * ln [ miR - 374 a - 5 p ] + ( f ) * ln [ miR - 182 - 5 p ] - ( g ) * ln [ miR - 345 - 5 p ] + ( h ) * ln [ miR - 361 - 3 p ] - ( i ) * ln [ miR - 130 b - 3 p ] - ( j ) * ln [ miR - 1299 ] - ( k ) * ln [ miR - 376 c - 3 p ] - ( l ) * ln [ miR - 326 ] ;$$
wherein:
(a)=any number from 23 and 33;
(b)=any number from 0.14 and 0.24;
(c)=any number from 2.5 and 7.5;
(d)=any number from 2 and 6;
(e)=any number from 4 and 9;
(f)=any number from 2 and 6;
(g)=any number from 0.5 and 5;
(h)=any number from 22 and 32;
(i)=any number from 1 and 5;
(j)=any number from 3 and 10;
(k)=any number from 4 and 11; and
(l)=any number from 6 and 14; and
wherein “[X]” refers to the amount of “X” in the biological sample and “Ln” represents the natural log;
wherein the human subject is identified as experiencing or at risk of experiencing an acute heart allograft rejection comprising an ACR if the ACR signature score is equal to or higher than about 65.

22-27. (canceled)

28. The method of claim 21, further comprising treating an acute heart allograft rejection by administering an immunosuppressive therapy to the human subject identified as having an ACR signature score that is equal to or higher than about 65.

29-33. (canceled)

34. A method of identifying a human subject experiencing or at risk of experiencing an acute heart allograft rejection comprising an antibody-mediated rejection (AMR) following a heart transplant comprising:
(i) obtaining a biological sample from the human subject, wherein the biological sample comprises RNA;
(ii) contacting the biological sample comprising the RNA with the panel of claim 17; and
(iii) determining an AMR signature score according to the following formula:
$$AMR signature score = 222.41 - ( a ) * ln [ miR - 23 a - 3 p ] - ( b ) * ln [ miR - 484 ] - ( c ) * ln [ miR - 340 - 5 p ] + ( d ) * ln [ miR - 193 a - 5 p ] - ( e ) * ln [ miR - 215 - 5 p ] + ( f ) * ln [ miR - 142 - 3 p ] - ( g ) * ln [ miR - 374 a - 5 p ] + ( h ) * ln [ miR - 1307 ] + ( i ) * ln [ miR - 185 - 3 p ] + ( j ) * ln [ miR - 4433 b - 3 p ] + ( k ) * ln [ miR - 130 b - 3 p ] + ( l ) * ln [ miR - 331 - 5 p ] + ( m ) * ln [ miR - 140 - 5 p ] + ( n ) * ln [ miR - 223 - 5 p ] + ( o ) * ln [ miR - 582 - 3 p ] + ( p ) * ln [ miR - 122 - 3 p ] + ( q ) * ln [ miR - 589 - 5 p ] ;$$
wherein:
(a)=any number from 23 to 28;
(b)=any number from 7 to 11;
(c)=any number from 1 to 6;
(d)=any number from 3 to 8;
(e)=any number from 6 to 11;
(f)=any number from 5 to 11;
(g)=any number from 1 to 5;
(h)=any number from 0.5 to 4;
(i)=any number from 5 to 11;
(j)=any number from 6 to 13;
(k)=any number from 3 to 8;
(l)=any number from 0.1 to 2;
(m)=any number from 1 to 4;
(n)=any number from 1 to 5;
(o)=any number from 0.5 to 4;
(p)=any number from 0.1 to 3; and
(q)=any number from 0.1 to 4; and
wherein “[X]” refers to the amount of “X” in the biological sample, and Ln represents the natural log;
wherein the human subject is identified as experiencing or at risk of experiencing an acute heart allograft rejection comprising an AMR if the AMR signature score is equal to or higher than about 65.

35-40. (canceled)

41. The method of claim 34, further comprising treating an acute heart allograft rejection by administering an immunosuppressive therapy to the human subject identified as having an AMIR signature score that is equal to or higher than about 65.

42-49. (canceled)

50. A kit comprising (A) or (B):
(A)(i) one or more RNA-hybridization probes, wherein at least one of the RNA-hybridization probes hybridizes to an miR selected from the group consisting of miR-30e-5p, let-7g-5p, miR-223-3p, miR-3615, miR-374a-5p, miR-182-5p, miR-345-5p, miR-361-3p, miR-130b-3p, miR-1299, miR-376c-3p, miR-326, and any combination thereof; and
(ii) instructions for measuring the level of a panel of miRs according to the method of claim 21; or
(B)(i) one or more RNA-hybridization probes, wherein at least one of the RNA-hybridization probes hybridizes to an miR selected from the group consisting of miR-23a-3p, miR-484, miR-340-5p, miR-193a-5p, miR-215-5p, miR-142-3p, miR-374a-5p, miR-1307, miR-185-3p, miR-443-3p, miR-130b-3p, miR-331-5p, miR-140-5p, miR-223-5p, miR-582-3p, miR-122-3p, miR-589-5p, and any combination thereof; and
(ii) instructions for measuring the level of a panel of miRs according to the method of claim 34.

51-101. (canceled)
